(19) United States
(12) Patent Application Publication
(10) Pub. No.: US 2025/0258152 A1
(43) Pub. Date: Aug. 14, 2025

(54) ADAPTIVE CONTROL AND ANAYSIS SYSTEM FOR SPIRITS PRODUCTION

(71) Applicant: QOMPLX LLC, Reston, VA (US)

(72) Inventors: Jason Crabtree, Vienna, VA (US); Richard Kelley, Woodbridge, VA (US); Jason Hopper, Halifax (CA); David Park, Fairfax, VA (US)

(21) Appl. No.: 18/809,270

(22) Filed: Aug. 19, 2024

Related U.S. Application Data
(63) Continuation-in-part of application No. 18/656,612, filed on May 7, 2024.
(60) Provisional application No. 63/551,328, filed on Feb. 8, 2024.

(51) Int. Cl. G01N 33/14; C12G 3/02; C12H 1/22; C12H 3/00

(52) U.S. Cl. CPC G01N 33/146; C12G 3/02; C12H 1/22; C12H 3/00

(57) ABSTRACT

Integrated intelligent system for beverage production, including distilled spirits and various brewed or fermented products, combining traditional methods with advanced technologies. System incorporates controlled fermentation unit, advanced distillation apparatus, and accelerated aging unit using novel methods like ultrasonic waves and thermal cycling. Real-time analysis system employs multiple sensors for continuous chemical composition monitoring throughout production process. Intelligent control mechanism optimizes processes and adjusts parameters dynamically. Chemical fingerprinting system enables precise quality control and authenticity verification. Flexible interface allows for customization of beverage profiles based on desired flavor characteristics and market demands. Specialized unit creates complex non- alcoholic alternatives. System adapts to different production scales and beverage types. Represents significant advancement in beverage production technology, offering unprecedented control, consistency, efficiency, and rapid product development across various beverage categories.

### CROSS-REFERENCE TO RELATED APPLICATIONS

Priority is claimed in the application data sheet to the following patents or patent applications, each of which is expressly incorporated herein by reference in its entirety: 18/656,612 63/551,328

### BACKGROUND OF THE INVENTION

### Field of the Art

The present invention is in the field of distilled spirit production and analysis, and in particular to the integration of advanced technologies for optimizing and accelerating the manufacturing process of high-quality alcoholic and non-alcoholic beverages.

### Discussion of the State of the Art

The production of high-quality distilled spirits, particularly aged varieties like whiskey and rum, has been a time-honored tradition for centuries. However, as global demand for premium spirits, beer, wine, fermented products, and non-alcoholic alternatives thereof continues to rise, traditional production methods are struggling to keep pace with changing preferences or suffer from inefficiencies. The lengthy aging process, typically ranging from 3 to 20 years (or more), ties up significant capital and inventory space, creating a bottleneck in production capacity and requiring immense capital. Moreover, the “Angel's Share”—the portion of spirit lost to evaporation during aging—represents a substantial economic loss, estimated at 2% per year in many climates. By 2025, the global spirits market is projected to reach $1.5 trillion, highlighting the urgent need for innovation in production methods to meet this growing demand without compromising, or indeed improving, quality.

Current solutions to accelerate spirit production and aging primarily focus on two approaches: alternative aging techniques and flavor additives. Alternative aging methods, such as the use of smaller barrels or wood chips, can speed up the maturation process but often fail to replicate the complexity of traditionally aged spirits. Flavor additives, while providing an attempt at a quick fix, are generally frowned upon in premium spirit production and can lead to inconsistent or artificial flavor profiles that are undesired by prospective consumers. Neither of these approaches adequately addresses the core challenges of production efficiency, quality consistency, and authenticity in spirit manufacturing to meet consumer demands or match premium traditional products on a chemical basis. This demand for innovation extends beyond traditional spirits to other beverage categories, including brewed or fermented beverages such as coffee or kombucha.

Furthermore, the rise of the craft spirits movement has led to increased experimentation and demand for unique flavor profiles and combinations of ingredients and techniques. Traditional production methods, with their long lead times, limit the ability of distillers to rapidly innovate and respond to changing consumer preferences or demands and volume needs. This has created a growing need for more flexible and responsive production systems that can produce high-quality, complex spirits in shorter timeframes based on present and near-term market demands and preferences.

The non-alcoholic spirits market is also experiencing rapid growth, projected to reach $39 billion in 2031. However, creating non-alcoholic alternatives that match the complexity and mouthfeel of traditional spirits remains a significant largely unaddressed challenge. Current methods often result in products that lack the depth and character of their alcoholic counterparts and often retain low levels of alcohol presence (most often referred to as 0.05% ABV limit).

Quality control and authenticity verification in the beer, wine and spirits industry also present ongoing challenges. The complexity of spirit composition makes it difficult to establish reliable markers of quality and maturity, leading to issues with counterfeit products and inconsistent quality across batches. Traditional sensory evaluation methods, while valuable, lack the objectivity and reproducibility needed for large-scale quality assurance. Additionally, premium products and vintage beverages may be faked and sold to unsuspecting collectors or consumers by unscrupulous actors.

What is needed is an integrated system that can accelerate the production of high-quality spirits and other beverages while maintaining or even enhancing flavor complexity and authenticity or producing specific needed chemical compounds which are differentiating. Such a system should optionally combine advanced fermentation and distillation techniques, innovative aging methods, real-time analytical and simulation modeling capabilities, and data-driven process optimization. Additionally, it should be flexible enough to produce both alcoholic and non-alcoholic spirits, beers, wines and other beverages with complex flavor profiles and provide objective measures for quality control and authenticity verification.

### SUMMARY OF THE INVENTION

The present invention introduces a novel system and method for analyzing and producing distilled spirits, beer, wine, brewed or fermented beverages and products, and non-alcoholic beverages using artificial intelligence and advanced technologies. This system combines traditional ingredient processing (manual and robotic) alongside distillation and fermentation methods with innovative techniques to accelerate the aging and flavor/compound development process while maintaining or enhancing the quality and complexity of the spirits.

Accordingly, the inventor has conceived and reduced to practice, a system and methods for producing distilled spirits using a combination of traditional and advanced technologies, including adaptive process optimization. The system includes a brewing and fermentation unit with controlled environmental conditions, a distillation apparatus combining traditional or vacuum distillation, an accelerated aging unit or reactor, real-time analytical instruments for chemical and particulate composition analysis, and an intelligent control system with simulation modeling and specialty physics and chemical machine learning models for process optimization. The system also incorporates a comprehensive chemical database of inorganic, organic, volatile, and comprehensive chemical compound profiles and a flexible production interface for customizing spirit profiles. The system also includes a database of critical process elements, ingredients, known existing products (e.g. historical wines, beers or spirits of value or interest), and may also integrate with customer relationship management software for direct to consumer “on demand” production modifications (or business to business equivalents where distribution is conducted, or required, to occur through ABCs or other retailer or distribution partners). This integrated approach allows for efficient production of high-quality spirits with reduced aging time, as well as the creation of custom flavor profiles and non-alcoholic alternatives closer to current consumer preferences and demands to improve capital efficiency, increase margins and value delivered to customers. The system is adaptable to various fermented products, including spirits, beer, wine, and condiments, each benefiting from the precise control and analysis capabilities.

According to a preferred embodiment, a system for producing distilled spirits is disclosed, comprising: a fermentation unit with controlled environmental conditions; a distillation apparatus combining traditional and vacuum distillation; an accelerated aging unit; real-time analytical instruments for chemical and particulate composition analysis; a control system utilizing artificial intelligence for process optimization; a database of chemical profiles; and a flexible production interface for customizing spirit profiles.

According to an aspect of an embodiment, a continuous flow system may be incorporated into the distillation and/or aging process.

According to another preferred embodiment, a method for producing distilled spirits is disclosed, comprising the steps of: fermenting a mash under controlled conditions optimized by adaptive control systems; distilling the fermented mash using a combination of traditional and vacuum distillation; accelerating the aging process using at least one novel aging technique which may involve additional chemical alterations, thermal cycling, pressure cycling, mixing or agitation, or recursive distillation; continuously monitoring the chemical composition of the spirit during production processes; adjusting production parameters in real-time based on intelligent analysis of monitoring data from an ongoing production run and persisting such data for future analysis and model or simulation development; and comparing the final product's chemical profile to a database of known profiles (e.g. via liquid or gas chromatography mass spectrometry, turbidity, viscosity, specific gravity, photometric analysis, electrometric analysis, thermal conductivity, density) for quality assurance, valuation/pricing, labeling, and scoring.

According to another preferred embodiment, a system for analyzing and authenticating distilled spirits is disclosed, comprising: a database of profiles for mature spirits; analytical instruments for generating a profile of a test spirit; a comparison algorithm for matching the test spirit's profile against the database; and an artificial intelligence subsystem for interpreting the comparison results and determining authenticity or maturity.

According to another preferred embodiment, a method for creating custom spirit flavor profiles is disclosed, comprising the steps of: inputting desired flavor characteristics into a flavor prediction model; receiving suggested production parameters from the model; producing a small batch of spirit using the suggested parameters; analyzing the produced spirit's chemical composition and flavor profile; feeding the analysis results back into the system for refinement; and iterating the process until the desired flavor profile is achieved with optional links to online data (e.g. wine or beer rating sites or data sets or retailer reviews) and crowd/expert sourced tagging and review information from tasters or consumers to support flavor prediction dataset and associated model(s).

### BRIEF DESCRIPTION OF THE DRAWING FIGURES

FIG. 1 is a block diagram illustrating exemplary adaptive control and analysis system architecture.

FIG. 2 is a block diagram illustrating exemplary intelligent data flow.

FIG. 3 is a block diagram illustrating an exemplary architecture of intelligent control system

FIG. 4 is a block diagram illustrating adaptive fermentation control system.

FIG. 5 is a method diagram illustrating the use of adaptive fermentation control system.

FIG. 6 is a block diagram illustrating dynamic distilling control and optimization system architecture

FIG. 7 is a method diagram illustrating the use of dynamic distilling control and optimization system.

FIG. 8 is a block diagram illustrating dynamic flavor prediction and control system architecture.

FIG. 9 is a method diagram illustrating the use of dynamic flavor prediction and control system.

FIG. 10 is a block diagram illustrating adaptive spirit aging system architecture.

FIG. 11 is a method diagram illustrating the use of the adaptive spirit aging system.

FIG. 12 is a block diagram illustrating comprehensive chemical fingerprinting system architecture.

FIG. 13 is a method diagram illustrating the use of comprehensive chemical fingerprinting system.

FIG. 14 is a block diagram illustrating alcohol removal system architecture.

FIG. 15 is a method diagram illustrating the use of alcohol removal system.

FIG. 16 is a block diagram illustrating real-time analysis and control system architecture.

FIG. 17 is a method diagram illustrating the use of real-time analysis and control system.

FIG. 18 illustrates an exemplary computing environment on which an embodiment described herein may be implemented, in full or in part.

### DETAILED DESCRIPTION OF THE INVENTION

The inventor has conceived, and reduced to practice, a system and methods for producing and analyzing distilled spirits, beer, wine and beverages using artificial intelligence and advanced process control technologies with enhancements including machine learning and simulation modeling. The system combines traditional ingredient processing, fermentation and distillation methods with innovative techniques to accelerate development of flavor compounds and flavor-active chemicals via individual and combined ingredient processing alongside fermentation or aging processes while maintaining or enhancing the quality and complexity of the spirits or beverage. This integrated approach allows for efficient production of high-quality spirits with precise reproducible production chains with the potential for closer to consumption production and reduced aging time, as well as the creation of custom flavor profiles and non- alcoholic alternatives to maximize consumer value.

The system combines traditional ingredient processing, fermentation and distillation methods with innovative techniques to accelerate development of flavor compounds and flavor- active chemicals via individual and combined ingredient processing alongside fermentation or aging processes while maintaining or enhancing the quality and complexity of the spirits, vinegars, or other products.

According to some embodiments, the system comprises ingredient processing and (optional) fermentation unit with controlled environmental conditions, adaptive spirit aging unit, real-time analytical instruments for chemical composition analysis, control system utilizing artificial intelligence for process optimization, database of chemical compound profiles and consumer preferences, and flexible production interface for customizing spirit/beverage profiles.

Customization of profiles may be at the chemical compound layer (e.g. a list of must have compounds like furfural for nutty notes of almond or walnut, hydroxymethyl for butter or caramel notes, vanillin, guaiacol for smoky or peppery notes, eugenol for clover or nutmeg or cinnamon, cyclotene for maple or caramel essence); via a model (e.g. an LLM) which allows a user to specify desired characteristics (e.g. “I'd like a Bourbon with rich notes of dark fruit, hints of vanilla, deep caramel, and toasted oak on the nose with a full body combining vanilla, chocolate and hazelnut with a touch of apple when tasted and a creamy finish that includes hints of honeyed apple”) via tasting notes or descriptions, or via a description of other product similarities (e.g. “Make me a bourbon as close to Pappy Van Winkle 23 as possible!”). The ingredient processing and fermentation unit allows for precise control of conditions to optimize the production of flavor precursors, while the distillation apparatus combines traditional distillation or rectification methods with optional vacuum distillation to capture a wider range of congeners.

According to an embodiment an adaptive spirit aging system is present. Adaptive spirit aging system may include technologies such as a dual walled stainless steel reactor with agitation, ultrasonic aging, rapid thermal cycling, or packed bed bioreactors. These techniques aim to replicate or accelerate the chemical reactions that occur during traditional barrel aging (or sequences of barrel aging such as oak to cherry to portwood). Real-time analytical instruments, which may include gas chromatography-mass spectrometry (GC-MS) systems, electrometric sensors, turbidity sensors, pH meters, and thermal analyzers continuously monitor the characteristics and chemical composition of the spirit during production processes. This data is fed into an adaptive control system, which uses machine learning algorithms to predict flavor outcomes and adjust production parameters in real-time. Since flavor perceptions are highly complex results of numerous molecules with non-linear and complex, and often concentration dependent, synergistic and antagonistic characteristics the system leverages specially trained models which are based on consumer feedback (e.g. this beer was very bitter with nice acidity and a bit of sweetness), expert tasters, and existing products (i.e. those with already known commercial success) and can solicit ongoing feedback from the user or request to “go out” for additional feedback or human perception testing to reduce its estimated uncertainty regarding a new molecule combination/concentration mix that is in an insufficiently mapped/modeled region. Predicted factors include elements like specific gravity, density, mouthfeel, taste, coloration, turbidity and smell which may influence ultimate consumer satisfaction and commercial value, or lack thereof. Similarly, system may optionally interact with “companion” food/drink database which stores chemical compounds and flavor profiles of other foods or drinks to aid in recommended pairings—enabling user to ask for an appropriate complement spirit or drink for a given meal or situation.

In an embodiment, an intelligent control system is present. It analyzes data from the ingredient processing, fermentation, distillation, and aging stages (note that system may use a process graph to represent the various stages and implement directed acyclic graph descriptions of such processes such as recurring distillation with ongoing chemical compound additions in various distillation and aging stages) to make informed decisions about process parameters and control other equipment (e.g. send a pulse to a peristaltic pump for dispensing a flavor additive into a vacuum distiller at certain time points). The system learns from each production run and resulting consumer feedback and chemical/characteristic profiling, continuously improving its ability to predict and achieve desired flavor profiles and developing an increasingly complete and certain map of the nonlinear flavor compound combinatorial/concentration space. This adaptive approach allows for consistent quality and the ability to create unique, custom spirits.

In an embodiment a database of chemical compound profiles is present. By comparing the final product's comprehensive chemical profile to known profiles of mature spirits, the system can objectively assess the quality and maturity of the produced spirit. This feature is particularly valuable for ensuring consistency across batches and detecting potential counterfeit products.

The flexible production interface allows for the customization of spirit profiles based on desired flavor characteristics. Users can input specific flavor goals, and the AI system will suggest production parameters to achieve those goals. This feature enables the creation of bespoke spirits tailored to specific preferences or market demands.

An important aspect of the system is its ability to produce non-alcoholic spirit alternatives with complex flavor profiles. This capability addresses the growing market demand for sophisticated non-alcoholic beverages that mimic the complexity and mouthfeel of traditional spirits. The system uses advanced techniques to remove or substitute with a synthetic replacement, alcohol (or other specific chemicals or compounds with specific intoxication or health or legal characteristics which are not desired) while preserving or recreating the desirable flavor compounds found in targeted alcoholic spirits or other beverages.

The method for producing distilled spirits using this system involves several key steps. First, ingredients are combined and mixed with water or other solvent, a resulting solution or mash is created and optionally fermented under controlled conditions optimized by the AI enhanced control system. The processed or fermented mash is then distilled using a combination of traditional and vacuum distillation techniques with optional ongoing compound additions during different (optionally recurring distillation stages). The resulting spirit undergoes accelerated aging using one or more novel aging techniques and may incorporate additional compounds or distillation loops. Throughout the production process, the system continuously monitors the chemical composition of the spirit and adjusts parameters in real-time based on AI analysis of the monitoring data to maximize the likelihood of expected compound and molecule development and concentration levels profiled for each process point. Directed acyclic graph based process maps may commonly link a set of input and stage action and stage output expectations (e.g. particular solution characteristics) that serve as localized objective functions for the control system during a given stage, enabling localized control optimization routines to run with a global optimization routine optionally supervising across all stages to maximize process-level goals (e.g. total cost, available resources, and a schedule against product quality).

Quality assurance is maintained by comparing the final product's characteristics including its comprehensive chemical profile to a database of known profiles or the synthetic target profile constructed from the compound-flavor models or specified by the user. Additionally, the system can generate a unique chemical fingerprint for each produced spirit, which can be used for authenticity verification where different degrees of invasive analysis can be specified and linked to the observables associated—such as which things can be sensed thru the bottle (e.g. IR spectroscopy) and which things require opening it (e.g. GCMS profiling). This feature is particularly valuable in combating counterfeiting and ensuring product consistency in retail environments for supply chain verification or collector validation. This analysis may be performed using a liquid sample, or nondestructively through the original container using multiwavelength spectroscopy, polychromators, ultrasonic analysis, or combinations of multiple spectrometers with differing levels of accuracy which may be known and disclosed with the product.

The system and methods described herein also include a novel approach to creating custom spirit flavor profiles. This process begins by inputting desired flavor characteristics into an adaptive flavor prediction model. The model then suggests production parameters to achieve these characteristics. Parametric studies may be done in this manner, such as comparing different yeasts with the resulting concentrations of yeast derived esters (e.g. ethyl hexanoate, ethyl octanoate, ethyl decanoate, or ethyl acetate). A small batch of spirit or beer or wine is typically produced using these suggested parameters, and its chemical composition and flavor profile are analyzed. System may also link to omics data about yeast and ingredients to directly aid in assessing particular preferences for the variety of corn or barley or hops or yeast. Omics data stored by the system may include genomics, proteomics, metabolomics, metagenomics, epigenomics, phonemics, and transcriptomics data pertaining to the ingredients. The analysis results are fed back into the system for refinement, and the process is iterated until the desired flavor profile is achieved. This leverages the flavor compound and concentration machine learning models for process and ingredient selection and prioritization or scoring.

The comprehensive chemical fingerprinting system for analyzing and authenticating distilled spirits is an integral part of the overall invention. It comprises a database of solution profiles including comprehensive chemical compound profiles for mature spirits, analytical instruments for generating a comprehensive chemical profile of a test spirit, a comparison algorithm for matching the test spirit's profile against the database, and an artificial intelligence subsystem for interpreting the comparison results and determining authenticity or maturity or pairings or flavors or smells or value.

The accelerated aging techniques employed in the system may include exposing the spirit to ultrasonic waves, mechanical agitation (e.g. centrifuges, baffles, impellers, static mixers or turbines), rapid thermal cycling, pressure cycling, or combinations thereof. These methods aim to simulate the effects of traditional barrel aging and different environments (e.g. under the ocean, in a shipping container, in a rickhouse in Kentucky vs in a rickhouse in Scotland) in a fraction of the historically required time. Computer controlled ultrasonic waves can increase the interaction between the spirit and additives (e.g. wood or other elements), while rapid pressure and thermal cycling can mimic aspects of the seasonal temperature fluctuations that contribute to flavor development in traditional aging.

The system and methods described herein find applications across various aspects of the beverage industry. They can be used for efficient production of high-quality beverages, development of new and unique flavor profiles, creation of non-alcoholic beverage alternatives, quality control and authenticity verification, and research into aging processes and flavor development.

Adaptive fermentation control system can be extended to optimize other aspects of spirit production, such as grain selection, yeast strain choice, and blending processes. By considering a wide range of variables and their interactions, the system can suggest optimal combinations to achieve desired flavor profiles or efficiency goals.

The database of comprehensive chemical profiles can be continuously expanded and refined as more spirits are analyzed. This growing database enhances the system's ability to accurately assess spirit quality and authenticity. It also provides valuable insights into the relationship between chemical composition and perceived flavor, which can inform future product development. The database encompasses a comprehensive “genome” of each spirit. This includes mapping all possible paths to create specific combinatoric outcomes, the outcomes themselves, and crucially, their links to human perception. The database can be continuously expanded and refined as more spirits are analyzed, enhancing the system's ability to accurately assess spirit quality, authenticity, and predicted flavor profiles. This growing database provides valuable insights into the complex relationships between chemical composition, production methods, and perceived flavor, informing future product development and allowing for unprecedented precision in spirit creation.

The system also serves as a powerful predictive tool for traditional aging methods. When a distiller places a spirit into a keg for aging, the optimal aging duration is typically uncertain, requiring regular sampling to determine when the spirit is ready for bottling. The claimed invention addresses this challenge by combining comprehensive chemical analysis with historical and predicted environmental data to accurately forecast the flavor development of a specific batch of spirits over time.

The process begins with a full chemical analysis of the newly barreled spirit, creating a baseline profile. This profile is then fed into the AI system, along with data on the barrel type, storage conditions, and environmental factors such as temperature and humidity fluctuations. The system also incorporates historical data on how similar spirits have developed under various conditions.

Using this information, the AI generates a predictive model of how the spirit's flavor profile will evolve over time. This model is continuously refined as the system receives updates on environmental conditions and periodic sampling data. The distiller can query the system at any time to get predictions on the current estimated flavor profile of the spirit, when the spirit will reach its optimal flavor profile for bottling, and how changes in storage conditions might affect flavor development.

This predictive capability allows distillers to optimize their aging process, plan production schedules more effectively, and even experiment with different aging conditions to achieve desired flavor profiles. It transforms the traditionally uncertain art of spirit aging into a more precise, data-driven science, while still respecting the craft and complexity of spirit production.

The flexible production interface can be integrated with market trend data and consumer preference information. This integration allows producers to quickly respond to changing market demands by adjusting their production parameters to create spirits that align with current trends or anticipated future preferences.

The system's ability to produce non-alcoholic spirit alternatives can be further enhanced by incorporating advanced flavor extraction and reconstruction techniques. These may include using supercritical fluid extraction, molecular distillation, or other cutting-edge technologies to isolate and preserve key flavor compounds from traditional spirits, which can then be used to create complex, non-alcoholic beverages. The system's precise control over chemical composition allows for the creation of drinks with unique sensory experiences that extend beyond flavor profiles. For instance, a beverage could be designed to taste like a favorite spirit, but also incorporate compounds that trigger a visual transformation upon opening.

As an example, the drink might be formulated to undergo a chemical reaction when exposed to air, causing it to fluoresce a brilliant magenta color. This striking visual effect would not alter the flavor profile, maintaining the integrity of the taste experience. Moreover, the system could be programmed to ensure that this effect decays quickly over time, creating a “limited edition” experience for consumers.

Other potential applications could include beverages that change color based on temperature, cocktails that emit a subtle aroma when stirred, or drinks that create a mild tingling sensation on the palate without the use of carbonation. These novel sensory effects, precisely controlled and safely implemented, offer beverage manufacturers unprecedented opportunities to create memorable, multisensory experiences for consumers.

By leveraging the system's capabilities in this way, manufacturers can produce beverages that not only taste unique but also provide an engaging, interactive experience. This approach could revolutionize the beverage industry, blurring the lines between drink and spectacle, and creating new categories of products that appeal to consumers seeking novel and Instagram- worthy experiences.

The system's future potential extends to revolutionizing beverage distribution and consumption, potentially evolving into an advanced “beverage synthesizer” capable of instantly mixing and dispensing custom drinks. This device could combine concentrated flavor extracts, spirits essences, and non-alcoholic bases on demand, allowing users to create personalized beverages or replicate specific branded products. Implementation could progress from centralized mass production to regional bottling facilities, then to on-site systems in bars or restaurants, and ultimately to home units, offering unprecedented customization and convenience in beverage creation.

The methods for creating custom spirit flavor profiles can be extended to include sensory panel feedback in the iterative refinement process. By combining objective chemical analysis with subjective human evaluations, the system can better align its predictions and recommendations with perceived flavor qualities.

Understanding the correlations between production parameters, chemical compositions, and resulting flavor profiles is crucial for the effective operation of this system. The adaptive learning component continuously learns from these relationships, improving its ability to predict outcomes and suggest optimal production strategies. This learning process enables the system to adapt to new ingredients, techniques, or market demands, ensuring its long-term relevance and effectiveness in the ever-evolving spirits industry.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to usage in one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing subsystems, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

### DEFINITIONS

The term “congeners” refers to the substances produced during fermentation other than ethanol, including various alcohols, aldehydes, esters, and acids, which contribute to the flavor and aroma of distilled spirits.

The term “cuts” refers to the different fractions of the distillate collected during the distillation process, typically divided into heads, hearts, and tails.

The term “heads” refers to the initial portion of the distillate that contains a high concentration of low-boiling point compounds, often including undesirable substances that are typically discarded or redistilled.

The term “hearts” refers to the main portion of the distillate that contains the desired ethanol and flavor compounds, which is typically kept for aging or further processing.

The term “tails” refers to the final portion of the distillate that contains higher-boiling point compounds, which may include both desirable and undesirable flavor components.

The term “proof” refers to a measure of alcoholic strength, where 1 degree of proof is equal to 0.5% alcohol by volume.

The term “column still” refers to a type of still consisting of vertical columns with multiple plates or trays, used for continuous distillation and capable of producing higher-proof spirits in a single pass.

The term “pot still” refers to a traditional type of still used in batch distillation, typically made of copper and consisting of a pot, swan neck, and condenser.

The term “reflux” refers to the process in distillation where vapor condenses and falls back into the still, increasing purity and alcoholic strength.

The term “foreshots” refers to the very first portion of the distillate, which contains highly volatile and often toxic compounds that are always discarded.

The term “feints” refers to the later part of the tails that are often collected separately and redistilled in a subsequent batch.

The term “continuous flow system” refers to a production method where the spirit moves continuously through the fermentation, distillation, or aging processes, as opposed to batch production.

The term “ultrasonic aging” refers to a technique that uses ultrasonic waves to increase the interaction between the spirit and wood elements, accelerating the aging process.

The term “rapid thermal cycling” refers to a technique that subjects the spirit to controlled temperature fluctuations to mimic the effects of seasonal changes in traditional barrel aging.

The term “packed bed bioreactor” refers to a system used in spirit aging where the liquid flows through a bed of solid particles (such as wood chips) to enhance flavor extraction and aging reactions.

The term “vacuum distillation” refers to a distillation technique performed under reduced pressure, allowing for lower temperature distillation and potentially capturing a different range of flavor compounds compared to traditional distillation.

The term “machine learning algorithms” refers to computational methods used in the system to analyze data, learn patterns, and make predictions or decisions without being explicitly programmed, including but not limited to deep neural networks, random forests, recurrent neural networks, and support vector machines.

The term “deep neural networks” refers to a type of machine learning algorithm composed of multiple layers of interconnected nodes, used in the system for complex pattern recognition and prediction tasks related to spirit production and flavor profiling.

The term “random forests” refers to an ensemble learning method that constructs multiple decision trees and outputs the mean prediction of the individual trees, used in the system for classification and regression tasks in spirit production.

The term “recurrent neural networks” refers to a class of artificial neural networks where connections between nodes form a directed graph along a temporal sequence, used in the system for analyzing time-series data in spirit production and aging processes.

The term “support vector machines” refers to supervised learning models used for classification and regression analysis, employed in the system for various prediction and optimization tasks in spirit production.

The term “artificial intelligence subsystem” refers to the component of the integrated intelligent control system that employs various machine learning algorithms and data analysis techniques to make decisions, predictions, and optimizations throughout the spirit production process.

The term “Bayesian subsystem” refers to a component that applies probabilistic modeling based on Bayes' theorem to analyze data and make predictions, particularly in the comprehensive chemical fingerprinting system.

The term “reinforcement learning” refers to a type of machine learning where an agent learns to make decisions by taking actions in an environment to maximize a reward, used in the system for optimizing production parameters over time.

The term “neural upsampler” refers to a neural network-based component used to enhance and recover information in the spirit's chemical profile, potentially used in the comprehensive chemical fingerprinting system or flavor prediction system.

The term “AI XAI” or “Explainable Al” refers to methods and techniques in artificial intelligence that enable human users to understand and trust the results and output created by machine learning algorithms. AI XAI aims to make the decision-making processes of complex AI systems transparent and interpretable to humans. This includes generating human-readable explanations for AI decisions, visualizing the decision-making process, providing confidence levels for predictions, and highlighting the most influential features in a decision.

The term “acetic acid bacteria” refers to a group of bacteria that can oxidize ethanol to produce acetic acid.

The term “fermentation starter” refers to microorganisms or cultures used to initiate fermentation including yeasts, bacteria, and fungi such as koji (Aspergillus oryzae).

### Adaptive Control and Analysis System Overview

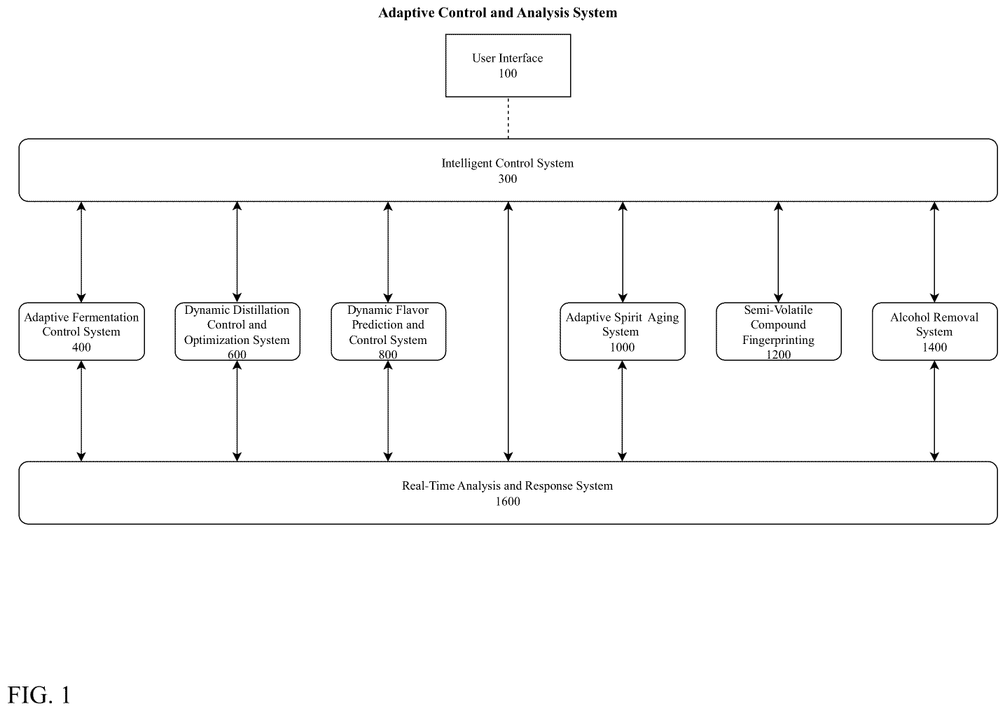

FIG. 1 is a block diagram illustrating an exemplary system architecture for an adaptive control and analysis system for spirits production, according to an embodiment. The term “adaptive control and analysis” refers to a system that combines multiple intelligent subsystems to provide a comprehensive approach to beverage production. These subsystems can exist in various forms, such as, for example, fermentation modeling, distillation processes, aging simulation and acceleration, and real-time monitoring. Examples of beverage types that can be produced by system can include, but are not limited to, whiskey, rum, vodka, gin, and non- alcoholic spirit alternatives.

Adaptive control and analysis system for spirits production will be described herein with respect to a use case directed to whiskey production, but the extent of the beverages which may be produced by system should not be construed or limited to only whiskey.

As shown, system comprises a plurality of subsystems including an intelligent control system 300, an adaptive fermentation control system 400, an advanced distillation system 600, a flavor modeling and prediction system 800, an adaptive spirit aging system 1000, a comprehensive chemical fingerprinting system 1200, an alcohol removal system 1400, a real- time monitoring system 1600, and a user interface 100 for direct control and guidance of the system. An adaptive fermentation control system 400 is designed to optimize the fermentation process, predicting outcomes based on various input parameters. An advanced distillation system 600 may have a traditional distillation system incorporated to work independently or in tandem to provide flexible distillation options, with the distillation system 600 incorporating modern techniques such as vacuum distillation. A flavor modeling and prediction system 800 utilizes intelligent algorithms to predict and optimize flavor profiles. An adaptive spirit aging system 1000 employs various techniques to speed up the maturation process. A comprehensive chemical fingerprinting system 1200 provides detailed chemical analysis for quality control and authenticity verification. An alcohol removal system 1400 enables the production of non- alcoholic alternatives. Finally, a real-time monitoring system 1600 continuously tracks various parameters throughout the production process, feeding data to other subsystems for optimal performance.

The intelligent control system 300 employs a sophisticated data integration framework to receive and process information from key subsystems in real-time. It interfaces directly with an adaptive fermentation control system 400, receiving continuous updates on various parameters which may include yeast activity, sugar content, and temperature profiles. From a distilling system 600, it may obtain data on vapor composition, temperature gradients, and pressure levels across different stages of the still, according to various embodiments. A dynamic flavor prediction and control system 800 may feed forward projected flavor profiles based on current production parameters, while also receiving feedback on actual outcomes to refine its models. An adaptive spirit aging system 1000 may provide data on wood interaction rates, chemical transformations, and maturation indicators, according to various embodiments. The comprehensive chemical compound fingerprinting system 1200 may supply detailed chemical composition data at various production stages, enabling the intelligent control system 300 to track the evolution of flavor compounds. For non-alcoholic variants, the alcohol removal system 1400 may transmit data on dealcoholization efficiency and flavor retention. The real-time analysis and response system 1600 acts as a critical feedback loop, continuously updating the intelligent control system 300 with analyzed data and automated responses from across all production stages. This intricate network of data flows allows the intelligent control system 300 to maintain a comprehensive, up-to-the-moment understanding of the entire production process, facilitating informed decision-making and proactive optimizations.

### Intelligent Data Flow for Adaptive Spirits Production

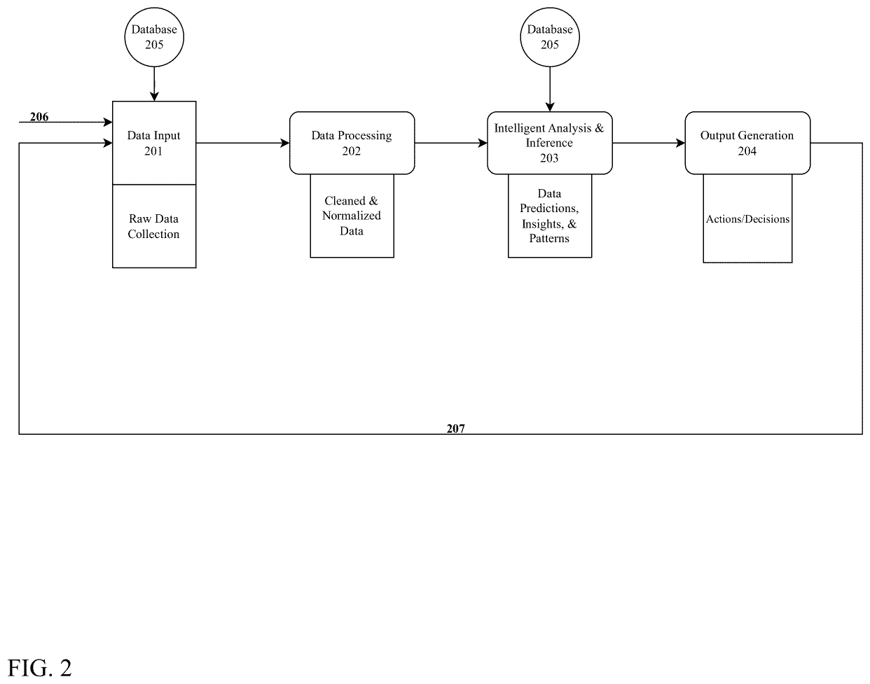

FIG. 2 is a block diagram illustrating exemplary intelligent data flow for which an embodiment described herein may be implemented, in full or in part. It presents a comprehensive overview of data flow in the adaptive control and analysis system for spirits production, illustrating the journey of information from its initial collection to the final output. The figure is structured into four main stages: data input 201, data processing 202, intelligent analysis and inference 203, and output generation 204. It also includes a database component 205 that interacts with multiple stages of the process.

The process starts with the data input 201 stage, where raw data 206 is collected from various sources throughout the production process. This may include but is not limited to real- time sensor data such as temperature readings from fermentation tanks and distillation columns, pressure measurements from the distillation apparatus, flow rates of liquids at different stages, chemical sensor data analyzing alcohol content and congener levels, and spectroscopic data from the comprehensive chemical fingerprinting system.

The data input 201 stage also draws from a comprehensive database 205. This database 205 may contain historical production data, reference flavor profiles, yeast strain characteristics, optimal fermentation and distillation parameters for different spirit types, regulatory information and constraints, and comprehensive chemical compound profiles for authentication. The integration of database 205 with real-time data 206 provides a rich, contextual foundation for the intelligent system's operations.

Following data input 201, the information progresses to the data processing 202 stage. Here, the raw data undergoes essential transformations to prepare it for intelligent analysis 203. This may involve cleaning the data by removing anomalous readings from faulty sensors, normalizing it by scaling temperature and pressure readings to standard ranges, and aligning time-series data from different stages of production. The processed data represents a refined version of the original input, optimized for analysis by the intelligent components of the system.

The core of the system lies in the intelligent analysis and inference 203 stage. This is where advanced algorithms and intelligent systems of this invention analyze the prepared data to extract meaningful insights and make decisions. For example, the intelligent systems may be neural networks predicting flavor profiles based on production parameters 400, reinforcement learning algorithms optimizing distillation processes 600, and Random Forests classifying spirit quality based on chemical composition 800. These intelligent systems may also interact with database 205 at this stage, leveraging historical data and reference information to inform their analyses and predictions. This combination of real-time data and historical context enables more accurate and nuanced decision-making.

The final stage in the linear flow is output generation 204. Here, the results of intelligent analysis 203 are transformed into actionable outputs. For example, the intelligent systems may make automated adjustments to fermentation 400 and distillation parameters 600 or predict final spirit flavor profiles 800. These outputs are carefully formatted to be easily interpretable by both the automated systems and human operators overseeing the production process.

A key feature of the figure is feedback loop 207, represented as an arrow connecting the output back to the input. This loop enables continuous learning and refinement. For example, feedback loop 207 may analyze the success of automated adjustments to improve control algorithms 300, or incorporate expert taster feedback to enhance flavor prediction accuracy 800. Importantly, this feedback is also used to update and enrich the database, ensuring that the system's knowledge base grows and improves over time.

The intelligent data flow illustrated in FIG. 2 is adaptable to various stages and processes within the adaptive control and analysis system for spirits production while maintaining a clear, high-level representation of data flow. It encapsulates the essential stages of intelligent data processing in spirit production, from raw sensor input to refined production decisions, and highlights the cyclical nature of system improvement through its feedback mechanism. The integration of a comprehensive database throughout the process ensures that the system leverages both real-time data and historical knowledge, enabling more informed and contextually aware decision-making in the spirit production process. This intelligent system architecture facilitates continuous optimization and adaptation, potentially leading to improved spirit quality, production efficiency, and consistency across various types of spirit production.

### Detailed Description of Intelligent Control System

Intelligent control system 300 is designed to dynamically manage and optimize the entire spirit production process, integrating data from various subsystems to make real-time adjustments and ensure consistent, high-quality output.

Intelligent control system 300 will be described herein with respect to whiskey production, but its capabilities extend to various types of spirits and non-alcoholic beverages.

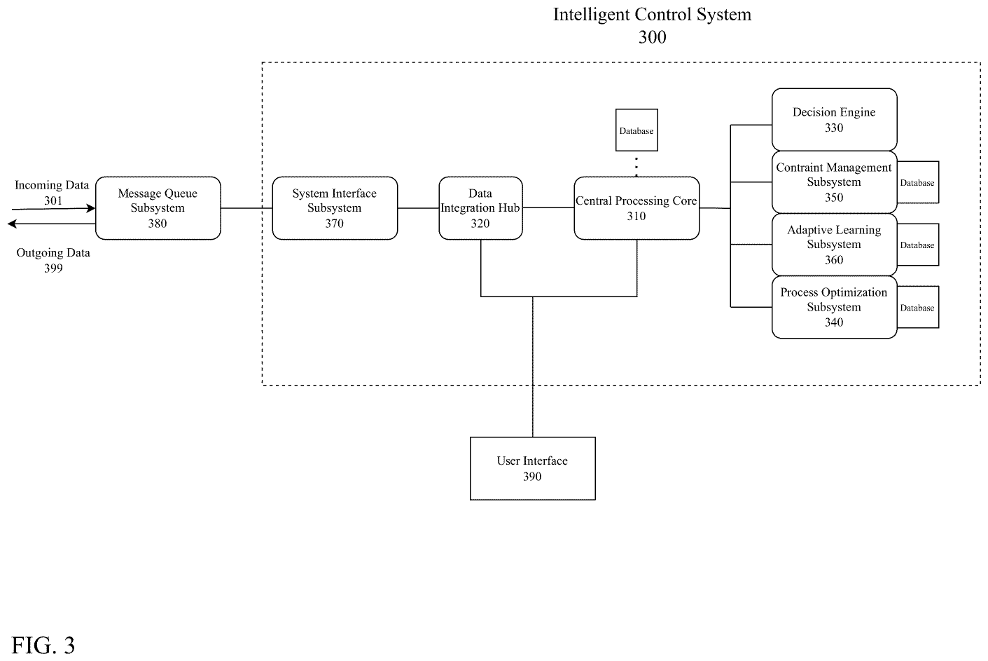

FIG. 3 is a block diagram illustrating an exemplary architecture of intelligent control system 300. As shown, system 300 comprises several interconnected components: central processing core 310, data integration hub 320, decision engine 330, process optimization subsystem 340, constraint management system 350, adaptive learning subsystem 360, system interface subsystem 370, message queue system 380, and interactive user interface 390.

The central processing core 310 is the primary computational unit of the intelligent control system. It utilizes a distributed computing architecture with GPU acceleration to handle complex calculations and decision-making in real-time. This architecture allows for parallel processing of multiple data streams and computationally intensive tasks. The core is equipped with high-performance CPUs for general-purpose computing and specialized GPUs optimized for machine learning and data processing tasks.

Central processing core 310 serves as the computational heart of the intelligent control system 300. It utilizes a distributed computing architecture with GPU acceleration to handle complex calculations and decision-making in real-time. This architecture allows parallel processing of multiple data streams and computationally intensive tasks. Central processing core 310 is equipped with high-performance CPUs for general-purpose computing and specialized GPUs optimized for machine learning and data processing tasks.

Central processing core 310 implements a multi-tiered caching system to minimize latency in data access and processing. This includes L1, L2, and L3 cache levels, with the L1 cache being closest to the processor for fastest access to frequently used data. Central processing core 310 also features dynamic frequency scaling, allowing it to adjust its clock speed based on workload demands, optimizing performance while managing power consumption.

For enhanced reliability, central processing core 310 incorporates error-correcting code ECC memory to detect and correct data corruption. It also employs a redundant array of independent disks RAID configuration for data storage, ensuring data integrity and providing failover capabilities.

Data integration hub 320 collects and harmonizes information from all integrated systems of the adaptive control and analysis system for spirit production. It employs a deep learning- based autoencoder network, trained on historical production data, to clean and normalize incoming data. The hub uses a federated learning approach to continuously improve its data processing capabilities while maintaining data privacy across different production facilities.

Data integration hub 320 is significantly affected by its interaction with the system interface subsystem 370 and the message queue system 380. This setup enables real-time data streaming, enhances scalability, ensures data consistency, and allows for asynchronous processing. The hub can selectively subscribe to relevant topics in the message queue, effectively filtering the data it receives and prioritizing critical information. This architecture also provides fault tolerance, as the message queue can retain data if the hub experiences issues, ensuring no information is lost during downtime.

Decision engine 330 analyzes the integrated data and generates actionable insights. It employs a hybrid system combining a knowledge-based expert system with a deep reinforcement learning model. The reinforcement learning model, based on a Proximal Policy Optimization PPO algorithm, is trained through simulated production runs and fine-tuned with real production data.

Process optimization subsystem 340 uses a multi-objective genetic algorithm (MOGA) to fine-tune each stage of the production process. The MOGA is initialized with expert-designed chromosomes representing different production parameter combinations and evolves over time based on the quality and efficiency of production outcomes.

Constraint management system 350 uses a combination of symbolic AI and neural- symbolic integration to ensure all decisions adhere to predefined constraints and regulatory requirements. It employs answer set programming for efficient constraint satisfaction, while a graph neural network helps identify potential constraint violations in proposed process adjustments.

Adaptive learning subsystem 360 utilizes a meta-learning framework based on Model- Agnostic Meta-Learning (MAML) to continuously improve the system's performance. It is pre- trained on a diverse set of spirit production scenarios and can quickly adapt to new production environments or spirit types with minimal additional data.

The system interface subsystem 370 manages interactions between the intelligent control system 300 and other integrated systems. It acts as a translator and router for communications, converting high-level commands from the central processing core into specific instructions for each subsystem. The module formats information into a standardized message format compatible with the message queue system.

Message queue system 380 facilitates asynchronous communication between components of the intelligent control system 300 and other integrated systems. It uses a publish-subscribe model, where the system interface subsystem ‘publishes’ messages to specific topics or channels within the queue, and other systems or components ‘subscribe’ to relevant topics. This setup allows for reliable, scalable, and fault-tolerant data exchange.

Interactive user interface 390 provides a sophisticated yet intuitive means for human operators to interact with the intelligent control system 300. It features a responsive, web-based dashboard that can be accessed from various devices. The interface employs advanced data visualization techniques, including real-time 3D rendering of production processes and interactive charts, allowing operators to monitor and analyze complex production data efficiently.

A natural language processing subsystem is integrated into interface 390, allowing operators to input queries and commands in plain language. This feature enhances ease of use and reduces the learning curve for new operators. Interface 310 also incorporates an explainable AI XAI component, which provides transparent explanations for the system's decisions and recommendations. This aids in building trust and understanding between the human operators and the AI system. An adaptive user profiling system personalizes the interface based on individual user preferences and expertise levels. This ensures that both novice and expert users can efficiently interact with the system, with information and controls presented in a manner most suited to each user's needs and capabilities.

### Adaptive Fermentation Control System Overview

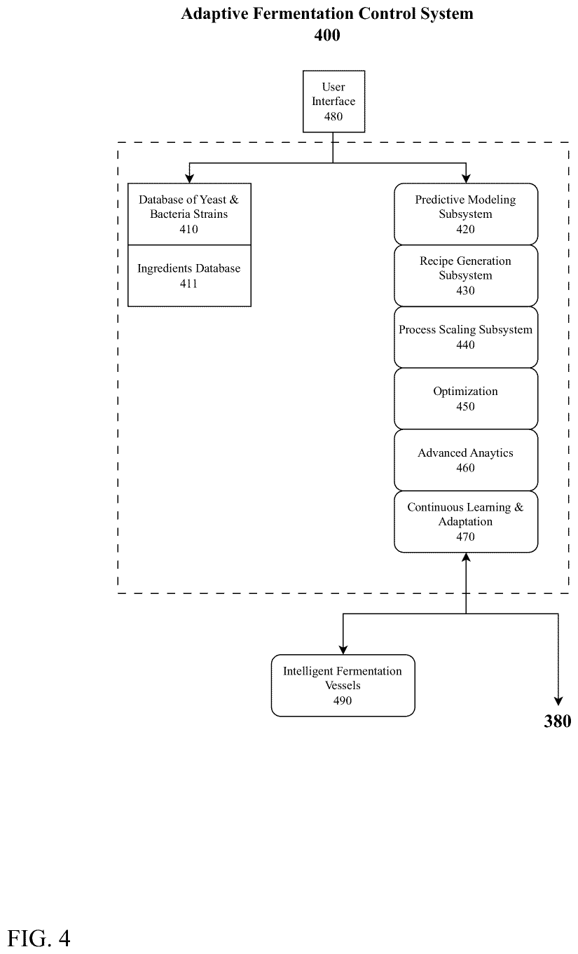

FIG. 4 is a block diagram illustrating adaptive fermentation control system 400. Adaptive fermentation control system 400 is designed to dynamically optimize and manage the fermentation process, a critical stage in spirit production that significantly influences the final product's flavor profile and quality.

Adaptive fermentation control system 400 will be described herein with respect to whiskey production, but its capabilities extend to various types of fermented beverages.

As shown, system 400 comprises several interconnected subsystems: database of yeast and bacteria strains 410, database of ingredients 411, predictive modeling subsystem 420, recipe generation subsystem 430, process scaling subsystem 440, optimization subsystem 450, advanced analytics subsystem 460, continuous learning and adaptation subsystem 470, user interface and reporting subsystem 480, and network of intelligent fermentation vessels 490. The system's flexibility extends to various fermentation processes, including vinegar production, where it can precisely control acetic acid bacteria activity alongside yeast fermentation.

Database of yeast and bacteria strains 410 contains comprehensive information on various yeast and bacteria strains, including their fermentation characteristics and flavor contributions. Ingredients database 411 stores detailed information on grains, adjuncts, and other fermentation inputs.

Predictive modeling subsystem 420 utilizes advanced machine learning algorithms to forecast fermentation outcomes and optimize processes. It employs deep learning models such as Long Short-Term Memory (LSTM) networks and Transformers to capture the complex, non- linear, and time-dependent nature of fermentation processes. This subsystem 420 also implements transfer learning techniques, allowing knowledge gained from one spirit type to be applied to others, thereby accelerating learning and improving predictions for new or less common spirits.

Predictive modeling subsystem 420 integrates reinforcement learning algorithms to continuously optimize fermentation parameters over time. By treating the fermentation process as a multi-step decision problem, the system learns to make sequences of decisions that maximize desired outcomes such as flavor profile, alcohol content, and efficiency. This approach allows the system to adapt to changing conditions and improve its strategies over time. An active learning component within predictive modeling subsystem 420 identifies which new experiments or data points would most improve the model's performance. This enables efficient use of resources by focusing on the most informative experiments, accelerating the system's learning and adaptation.

Recipe generation subsystem 430 creates optimal fermentation recipes based on desired outcomes. It employs generative models such as Variational Autoencoders (VAEs) and Generative Adversarial Networks (GANs) to generate new recipe ideas based on learned patterns from successful fermentations. Recommender systems are integrated to suggest combinations of ingredients and parameters based on similarity to successful past recipes. This subsystem also incorporates the flavor prediction system's outputs to align fermentation recipes with desired final flavor profiles.

Process scaling subsystem 440 ensures that fermentation processes can be effectively scaled up or down, maintaining consistency across different production volumes. It uses machine learning models trained on historical scaling data to predict and compensate for changes in fermentation dynamics at different scales.

Optimization subsystem 450 continuously refines the fermentation process based on real- time data and historical outcomes. It utilizes reinforcement learning to optimize fermentation processes over time, learning from the outcomes of different parameter choices. Genetic algorithms are employed to explore a wide range of possible fermentation recipes and conditions, evolving towards optimal solutions. Bayesian optimization techniques are used for fine-tuning fermentation parameters, especially when the relationship between parameters and outcomes is complex.

Advanced analytics subsystem 460 implements anomaly detection algorithms to identify potential issues early in the fermentation process. It uses multivariate analysis to understand complex interactions between fermentation parameters and develops a flavor precursor prediction model based on fermentation conditions. This subsystem also creates a digital twin of the fermentation process for simulation and prediction, enabling “what-if” scenario planning and predictive maintenance for fermentation equipment.

Continuous learning and adaptation subsystem 470 implements a feedback loop that incorporates tasting panel results to refine the model. It develops an automated experimentation system to continuously test and improve fermentation strategies. This subsystem also utilizes federated learning techniques to share insights across multiple production facilities while maintaining data privacy, allowing the system to learn from a wider range of experiences without compromising proprietary information.

User interface and reporting subsystem 480 develops a dashboard for real-time monitoring of fermentation status and predictions. It creates automated reports on fermentation performance and outcomes and implements interactive tools for fermentation scenario planning.

Adaptive fermentation control system 400 also incorporates a network of intelligent fermentation vessels 490. These vessels are equipped with an array of sensors and actuators, allowing for real-time monitoring and control of the fermentation process. Each vessel in the 490 subsystem is capable of independently adjusting temperature, agitation rate, and other critical parameters based on instructions from the other intelligent subsystems.

Intelligent fermentation vessel subsystem 490 continuously feeds data to the predictive modeling subsystem 420 and the advanced analytics subsystem 460. This data includes real-time measurements of temperature, pH, dissolved oxygen, sugar content, and alcohol concentration. In return, these intelligent subsystems send control signals back to the vessels, dynamically adjusting conditions to optimize the fermentation process.

For example, if the predictive modeling subsystem 420 anticipates a suboptimal fermentation trajectory based on current conditions, it can instruct the intelligent fermentation vessels 490 to adjust temperature or introduce nutrients to correct the course. Similarly, if the advanced analytics subsystem 460 detects an anomaly in one vessel, it can trigger automated corrective actions in that specific vessel while alerting the system operators through the user interface 480.

Process scaling subsystem 440 works closely with intelligent fermentation vessel subsystem 490 to ensure that recipes can be accurately scaled across different vessel sizes. It takes into account the specific characteristics of each vessel, such as heat transfer rates and mixing dynamics, to maintain consistency in fermentation outcomes regardless of batch size.

Network of intelligent fermentation vessels 490 forms the physical core of the adaptive fermentation control system 400. Each vessel is equipped with an array of advanced sensors for real-time monitoring of critical fermentation parameters such as temperature, pH, dissolved oxygen, sugar content, and alcohol concentration. The vessels also feature precision-controlled actuators for adjusting fermentation conditions, including temperature control systems, automated nutrient dispensers, and programmable agitators. These vessels are interconnected via a secure, low-latency network, allowing for coordinated control and data sharing across the entire fermentation process. The intelligent fermentation vessels 490 are designed with modular, scalable architecture, enabling easy expansion or reconfiguration of the fermentation capacity. They also incorporate self-diagnostic capabilities for predictive maintenance, ensuring optimal performance and minimizing downtime. The integration of these intelligent vessels with the adaptive control and analysis system for spirit production allows for unprecedented precision in fermentation management, enabling real-time adjustments and optimizations that were previously impossible in traditional fermentation setups.

In operation, adaptive fermentation control system 400 begins with the user inputting initial parameters through user interface 480, including the desired flavor profile, production goals, and grain type. Based on these inputs, the system suggests optimal yeast and bacteria strains from database 410, and the user selects the desired microorganisms.

Predictive modeling subsystem 420 then runs a simulation of the fermentation process using the selected parameters. System presents the predicted outcomes to the user through interface 480, including expected flavor precursors, alcohol content, and other relevant characteristics of the fermented product.

If these predicted outcomes are satisfactory, recipe generation subsystem 430 creates a detailed fermentation recipe, which can be scaled by subsystem 440 as needed, and fermentation is initiated in intelligent fermentation vessels 490. However, if the outcomes are unsatisfactory, the user can adjust parameters, or optimization subsystem 450 may suggest modifications to improve results.

Throughout the fermentation process, the system continuously monitors conditions using real-time sensor data from intelligent fermentation vessels 490, which is fed into predictive modeling subsystem 420 and advanced analytics subsystem 460. These subsystems work in tandem to detect anomalies, predict outcomes, and suggest real-time adjustments to optimize the process.

Continuous learning and adaptation subsystem 470 ensures that the system improves over time by incorporating feedback from each fermentation run, including post-production taste tests and quality assessments. This allows the system to refine its models and strategies continuously.

The adaptive fermentation control system 400 is designed to handle a variety of fermentation processes, from traditional yeast fermentation to more specialized processes using alternative fermentation starters such as koji, demonstrating the system's versatility across diverse beverage production methods. System's 300 flexibility extends to the production of non- alcoholic fermented beverages such as kombucha, where precise control of both yeast and bacterial fermentation is crucial for achieving desired flavors and health-promoting properties.

Adaptive fermentation control system 400 is designed to work in close coordination with intelligent control system 300. Intelligent control system 300 provides high-level directives and optimization goals to the adaptive fermentation control system 400 based on its analysis of the entire production process. In turn, adaptive fermentation control system 400 sends real-time fermentation data and performance metrics back to intelligent control system 300 through message queue system 380. This bidirectional flow of information allows for dynamic adjustments to fermentation parameters based on broader production goals and constraints. For example, if intelligent control system 300 determines that a particular flavor profile is needed based on current market demands, it can instruct adaptive fermentation control system 400 to adjust its parameters accordingly. Adaptive fermentation control system 400 then implements these changes while continuously monitoring and optimizing the fermentation process within the new parameters.

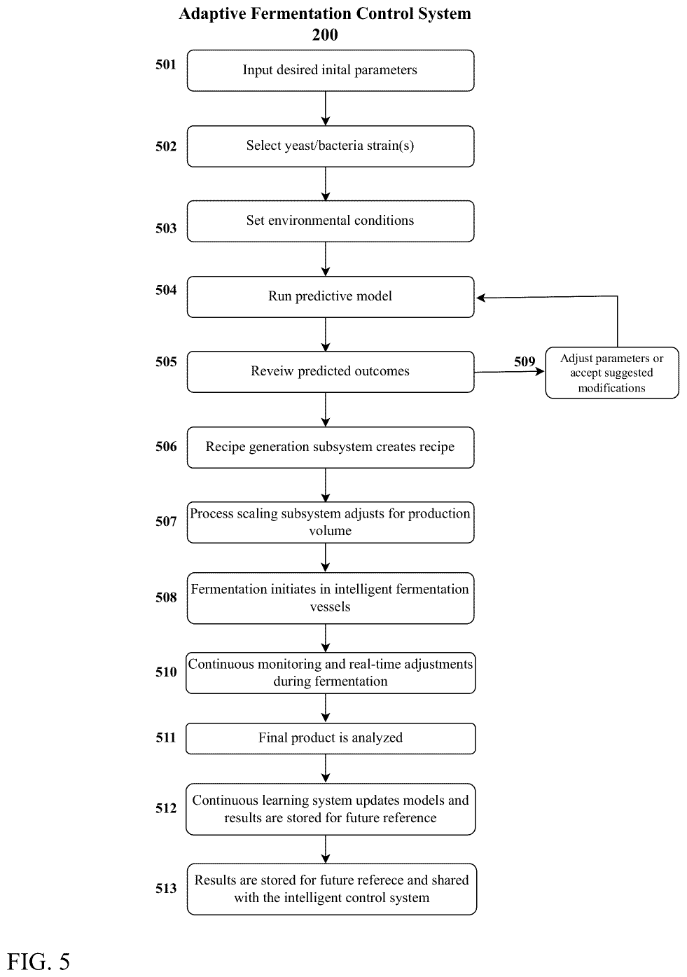

FIG. 5 is a method diagram illustrating the use of adaptive fermentation control system 400. The process begins with the user inputting initial parameters 501, including the desired flavor profile, production goals, and grain type. These inputs are processed by both adaptive fermentation control system 400 and intelligent control system 300. Based on these inputs, system 400 suggests optimal yeast and bacteria strains from database 210, and the user selects the desired microorganisms 502. System 400 then sets or suggests environmental conditions such as temperature, pH, and nutrient levels, along with distilling parameters 503, taking into account broader production goals provided by intelligent control system 300. Predictive modeling subsystem 420 runs a simulation of the fermentation process using the selected parameters 504. System presents the predicted outcomes to the user 505, including expected flavor precursors, alcohol content, and other relevant characteristics of the fermented product.

If these predicted outcomes are satisfactory, recipe generation subsystem 430 creates a detailed fermentation recipe 506, which is then scaled by subsystem 440 as needed for the production volume 507. Fermentation is then initiated in intelligent fermentation vessels 490 508. However, if the outcomes are unsatisfactory, the user can adjust parameters, or optimization subsystem 450 may suggest modifications to improve results 509, and the process returns to the simulation step 504. Throughout the fermentation process, system 400 continuously monitors conditions using real-time sensor data from intelligent fermentation vessels 290 510. Predictive modeling subsystem 420 and the advanced analytics subsystem 460 work in tandem to detect anomalies, predict outcomes, and suggest real-time adjustments to optimize the process. This real-time data is also shared with intelligent control system 300 through the message queue system 380.

Once the fermentation process completes, system 400 analyzes the final product 511. Continuous learning and adaptation subsystem 470 then updates its models based on the results 512, incorporating feedback from post-production taste tests and quality assessments. Finally, the results are stored for future reference 514 and shared with intelligent control system 300 for holistic process optimization. This process allows for a highly controlled, predictable, and optimized fermentation, leveraging the power of deep learning techniques to produce consistent, high-quality fermented products for spirit production. The adaptive nature of the system ensures continuous improvement over time, refining its models and strategies with each fermentation run.

This enhanced adaptive fermentation control system allows for a highly controlled, predictable, and optimized fermentation process, leveraging the power of advanced deep learning techniques to produce consistent, high-quality fermented products for spirit production. It also seamlessly integrates with other subsystems of the larger adaptive control and analysis system for spirit production, particularly the intelligent control system 300, ensuring a holistic approach to spirit creation.

### Dynamic Distillation Control and Optimization System Overview

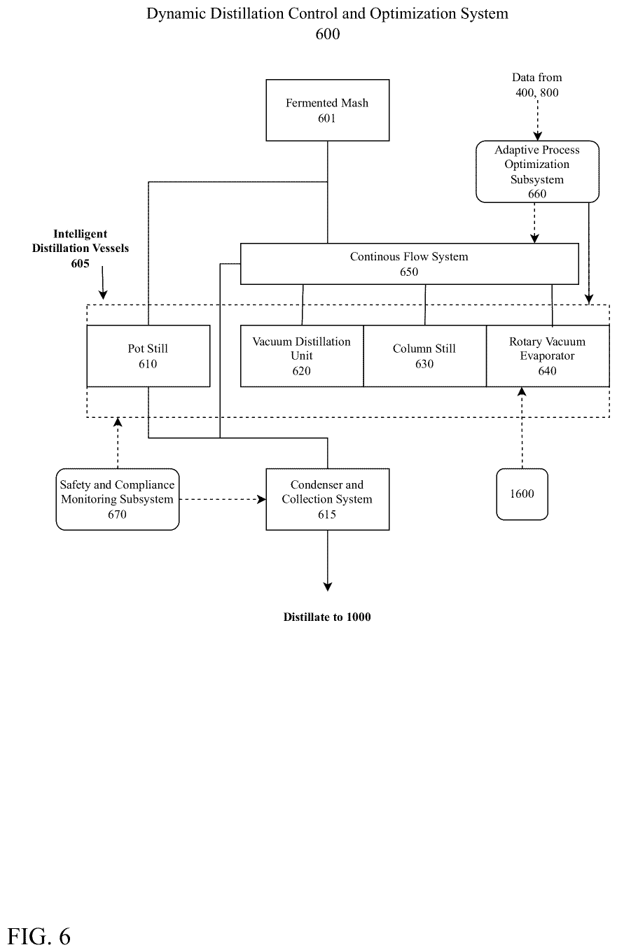

FIG. 6 is a block diagram illustrating an exemplary architecture of dynamic distillation control and optimization system 600. Dynamic distillation control and optimization system 600 is designed to provide greater flexibility in extracting and preserving flavor compounds to produce high-quality spirits with precise flavor profiles while optimizing energy efficiency and ensuring regulatory compliance.

Dynamic distillation control and optimization system 600 will be described herein with respect to whiskey production, but its capabilities extend to various types of spirits and scales of production.

As shown, system 600 comprises several interconnected components: network of intelligent distillation vessels 605, pot still system 610, condenser and collection system 615, vacuum distillation unit 620, modified column still 630, rotary vacuum evaporator 640, continuous flow control system 650, adaptive process optimization subsystem 660, and safety and compliance monitoring subsystem 670. The system 600 is designed with modularity and scalability in mind; the components can be used, interconnected, or omitted entirely depending on the user's desired methods, output, and production scale.

The network of intelligent distillation vessels 605 forms the core of the system, equipped with advanced sensors and actuators for precise control of distillation parameters. These vessels can be configured for both batch and continuous distillation processes, adapting to production needs. The pot still system 610, vacuum distillation unit 620, modified column still 630, and rotary vacuum evaporator 640 offer a range of distillation methods, each preserving different flavor compounds and allowing for precise control over the distillation process.

Continuous flow control system 650 implements a continuous distillation process for increased efficiency. This system uses peristaltic pumps for precise control of flow rates throughout the distillation process. It manages the flow of the fermented mash into the distillation apparatus, the movement of liquid and vapor between different stages of distillation, and critically, the flow of vapor into the condenser 615. Continuous flow system 650 is directly connected to condenser 615, ensuring a steady and controlled flow of vapor for condensation. This connection allows for real-time adjustments to the distillation rate based on feedback from the condenser, such as temperature differentials and condensate flow rates. The system 650 also controls the flow of the distillate from the condenser to the collection vessels, enabling precise fractionation of the distillate. By maintaining a constant, optimized flow throughout the entire distillation process, from mash input to final spirit collection, the continuous flow control system 650 plays a crucial role in ensuring consistency and quality in the final product.

Adaptive process optimization subsystem 660 employs a sophisticated deep learning approach, combining multiple machine learning techniques to optimize the distillation process. It is initially trained on a large dataset of historical production data, establishing a baseline understanding of the distillation process across various spirit types and production scales. This training is augmented with data from high-fidelity simulated distillation runs, allowing the system to explore a wide range of scenarios without the constraints of physical production.

The core of adaptive process optimization subsystem 660 consists of deep neural networks (DNNs) that model the complex, non-linear relationships between distillation parameters (such as temperature profiles, pressure levels, and flow rates) and output quality metrics (including flavor profiles and chemical composition). These DNNs are complemented by reinforcement learning (RL) agents, which optimize real-time decision-making during the distillation process. The RL agents learn through interaction with both simulated and real production environments, continuously refining their strategies to balance multiple objectives such as flavor accuracy, energy efficiency, and yield.

Genetic algorithms (GAs) are employed to explore the vast parameter space of possible distillation configurations. Each “individual” in the GA represents a specific combination of distillation parameters, with the fitness function evaluating these combinations based on simulated or historical outcomes. This approach allows the system 600 to discover novel, optimized parameter combinations for different spirit types and production goals.

The system 660 implements online learning algorithms to continuously update its models based on new production data. This allows the deep learning model to adapt to gradual changes in equipment performance, ingredient characteristics, or environmental conditions. Additionally, transfer learning techniques are used to quickly adapt pre-trained models to new spirit types or production scales, significantly reducing the data required to achieve good performance on new tasks.

Through this multi-faceted, continual learning approach, adaptive process optimization subsystem 660 constantly improves its performance with each production run. It can swiftly adapt to different spirit types and production scales, ensuring optimal distillation processes across a wide range of scenarios. This sophisticated deep learning integration places the system at the forefront of distillation technology, enabling unprecedented levels of control, efficiency, and quality in spirit production.

Safety and compliance monitoring subsystem 670 ensures that all operations adhere to regulatory requirements and safety standards, automatically documenting the process for compliance purposes. Safety and compliance monitoring subsystem 670 plays a crucial role in ensuring that all distillation operations adhere to regulatory requirements and safety standards. This subsystem integrates advanced sensors, real-time data analysis, and automated documentation to provide comprehensive oversight of the distillation process. It continuously monitors critical safety parameters such as temperature, pressure, and vapor concentrations using a network of high-precision sensors strategically placed throughout the distillation apparatus, including the condenser and collection system. If any parameter approaches or exceeds predefined safety thresholds, the system immediately triggers alerts, which can be visual alarms, audible warnings, or automated notifications sent to operators and supervisors. In case of severe deviations from safe operating conditions, the subsystem can initiate emergency shutdown procedures to prevent accidents or equipment damage.

System 670 maintains an up-to-date database of relevant regulations and industry standards, continuously cross-referencing operational parameters against these requirements to ensure compliance. All operational data, including safety-related events and compliance checks, are automatically logged and time-stamped, creating a comprehensive audit trail for regulatory inspections and internal quality assurance. By analyzing patterns in operational data, the subsystem can predict potential equipment failures or maintenance needs before they become safety issues, including those related to the condenser and collection system.

Robust user authentication and access control measures are implemented to ensure that only authorized personnel can make changes to critical system parameters. System 670 also monitors environmental factors such as air quality and effluent composition to ensure compliance with environmental regulations. While maintaining independence for safety reasons, it communicates with adaptive process optimization subsystem 660 to ensure that optimization decisions do not compromise safety or compliance. Regular self-diagnostic checks are performed to ensure its own sensors and systems are functioning correctly, alerting operators to any potential issues with the safety monitoring system itself.

System 600 also incorporates condenser and collection system 615. This component is crucial for efficiently cooling and condensing the vapor produced during distillation, as well as for properly separating and collecting different fractions of the distillate. The condenser utilizes a counter-current heat exchange design for maximum efficiency, with the ability to precisely control coolant flow rates and temperatures. This allows for fine-tuning of the condensation process to optimize flavor compound retention. The collection system employs multiple collection vessels with automated switching mechanisms, enabling precise cuts between foreshots, heads, hearts, and tails fractions. Each collection vessel is equipped with real-time monitoring of volume, alcohol content, and key flavor compounds, providing immediate feedback to the adaptive process optimization subsystem 660. The entire condenser and collection system is integrated with the network of intelligent distillation vessels 605, allowing for seamless coordination and optimization of the entire distillation process.

In operation, the distillation process begins as fermented mash 601 enters the system through an intake port, where it is immediately analyzed by sensors for composition and alcohol content. Continuous flow control system 650 then directs the mash to the appropriate distillation vessel within network of intelligent distillation vessels 605. Depending on the desired spirit profile and the mash characteristics, the system may route the mash to pot still system 610 for batch distillation, or to modified column still 630 for continuous distillation. In cases where lower temperature distillation is preferred, the mash may be directed to vacuum distillation unit 620. As the mash is heated and vaporized, the resulting vapor is channeled through the system, potentially passing through multiple distillation stages for further refinement. The vapor eventually reaches condenser 615, where it's cooled and converted back into liquid form. This distillate is then precisely divided into different fractions (foreshots, heads, hearts, and tails) by the collection system, with the hearts typically forming the main product. Throughout this process, rotary vacuum evaporator 640 may be employed for final refinement or for creating specialty spirits. At each stage, adaptive process optimization subsystem 660 analyzes real-time data to make adjustments, ensuring the final product meets the desired specifications.

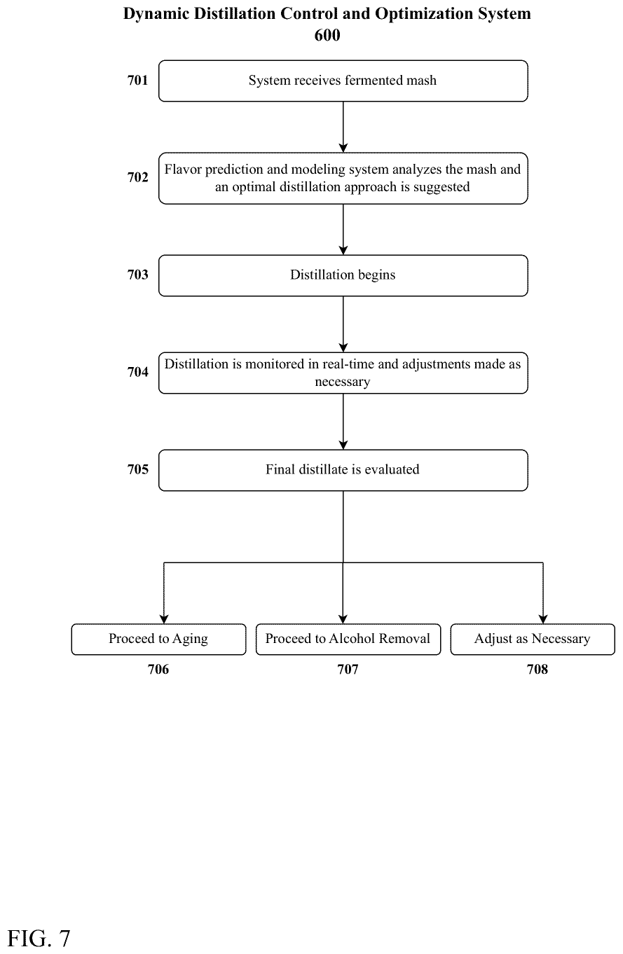

FIG. 7 is a method diagram illustrating the use of dynamic distillation control and optimization system 600. The operation begins with receiving the fermented mash 701 from adaptive fermentation control system 400. Flavor prediction and modeling system 800 analyzes fermented mash 702, and in conjunction with adaptive process optimization subsystem 660, suggests the optimal distillation approach 703. This may involve any combination of the distillation components, configured for either batch or continuous operation.

As distillation begins, real-time monitoring system 1600 continuously monitors the chemical composition of the vapor and distillate. The adaptive process optimization subsystem 660 adjusts distillation parameters in real-time 704 based on the instrument data to maintain the desired flavor profile while optimizing energy use. This may involve altering temperature, pressure, or flow rates in the various components, including the pot still system 610, vacuum distillation unit 620, modified column still 630, and condenser 615. If needed, the distillate may be routed through multiple distillation stages to achieve the desired composition.

System then evaluates the final distillate composition 705. If it meets the target profile, it

proceeds to the next production stage 706 707. If adjustments are needed, the process may be repeated with modified parameters 708. Upon completion, the system logs the production data, which is used to refine future distillation processes and inform adaptive fermentation control system 400 for optimizing subsequent fermentation runs.

This process allows for a highly controlled, flexible, and efficient distillation, enabling the production of a wide range of spirit profiles with consistency and precision. The integration of adaptive process optimization, intelligent distillation vessels, advanced monitoring systems, and safety and compliance monitoring subsystem 670 ensures that the distillation process continually improves, adapting to different spirits and production scales while maintaining quality, efficiency, and regulatory compliance. The system's ability to seamlessly switch between batch and continuous distillation, coupled with its energy optimization features, provides unprecedented flexibility and sustainability in spirit production.

### Dynamic Flavor Prediction and Control System Overview

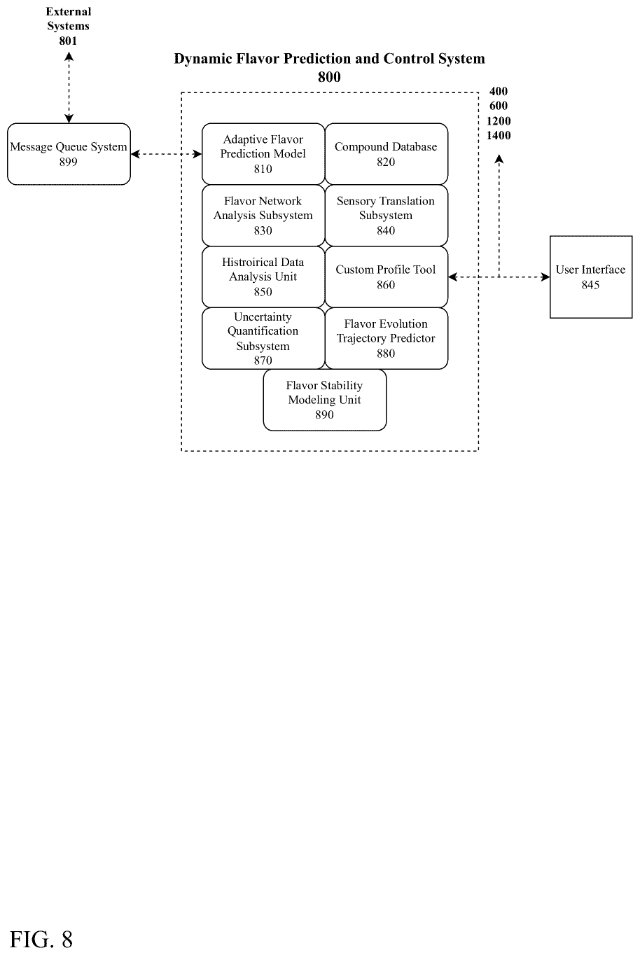

FIG. 8 is a block diagram illustrating an exemplary architecture of dynamic flavor prediction and control system 800. Dynamic flavor prediction and control system 800 is designed to forecast, analyze, optimize, and actively control flavor profiles in beverage production, playing a crucial role in achieving desired taste outcomes while adapting to real-time changes during production.

Dynamic flavor prediction and control system 800 will be described herein with respect to whiskey production, but its capabilities extend to various types of spirits and non-alcoholic products and beverages.

As shown, system 800 comprises several interconnected components: adaptive flavor prediction model 810, compound database 820, flavor network analysis subsystem 830, sensory translation subsystem 840, historical data analysis unit 850, custom profile development tool 860, a user interface 865, uncertainty quantification subsystem 870, flavor evolution trajectory predictor 880, flavor stability modeling unit 890, and message queue subsystem 899.

Adaptive flavor prediction model 810 utilizes a combination of machine learning algorithms to predict and optimize flavor outcomes based on production parameters. Principal Component Analysis (PCA) is used as a preprocessing step to handle the high-dimensional nature of flavor data. By reducing the dimensionality of the data while preserving its essential characteristics, PCA helps in visualizing flavor relationships and can improve the performance of other models in the system.

Regression models, particularly ensemble methods like Random Forests, are employed to predict continuous outcomes such as the intensity of specific flavor notes or concentrations of flavor compounds. These models are trained on historical production data, with the input features being production parameters (e.g., fermentation temperature, yeast strain, distillation cut points) and the target variables being measured flavor compound concentrations or sensory panel ratings. The Random Forest algorithm creates multiple decision trees and aggregates their predictions, which helps in handling complex interactions between input variables and reduces overfitting.

Deep Neural Networks (DNNs) are used to capture complex, non-linear relationships between production parameters and overall flavor profiles. These networks consist of multiple layers of interconnected nodes, each applying non-linear transformations to the input data. The DNNs are trained using backpropagation and gradient descent algorithms, with the network weights adjusted to minimize the difference between predicted and actual flavor profiles. The training data consists of historical production parameters and corresponding detailed flavor analyses.

Recurrent Neural Networks (RNNs), specifically Long Short-Term Memory (LSTM) networks, are applied to model time-dependent processes like fermentation and aging. These networks are designed to capture long-term dependencies in sequential data, making them ideal for predicting how flavors will evolve over time under different conditions. The RNNs are trained on time-series data from past production runs, learning to predict future flavor states based on current conditions and past trends.

Support Vector Machines (SVMs) are utilized for classification tasks within the system. For instance, they might predict whether a spirit will meet certain flavor criteria or fall into specific flavor categories based on its production parameters. SVMs are trained on labeled data from past productions, learning to find the optimal hyperplane that separates different flavor categories in a high-dimensional space.

Model 810 integrates these models to provide comprehensive flavor predictions and control strategies. For example, it may use PCA to preprocess the data, then feed the results into a combination of regression models and DNNs to predict specific flavor attributes. RNNs could then model how these flavors would develop over time, while SVMs classify the resulting flavor profile into predefined categories. This multi-model approach allows the system to capture different aspects of the flavor prediction and control task, from specific compound concentrations to overall flavor profiles and their development over time.

Compound database 820 maintains comprehensive information on flavor compounds, their origins, and sensory characteristics, including data on esters, aldehydes, phenols, and other flavor-active molecules found in various beverages, including spirits, wines, and brewed or fermented products such as coffee or kombucha.

Flavor network analysis subsystem 830 utilizes concepts from network theory to understand interactions between flavor compounds and predict how combinations of compounds will influence overall flavor profiles. This model treats flavor compounds as nodes in a complex network, with the interactions between compounds represented as edges. This approach allows for the analysis of how combinations of compounds contribute to the overall flavor experience, beyond what individual compound concentrations might suggest, applicable to a wide range of beverages including spirits, wines, and brewed or fermented products. The model builds a network based on the co-occurrence of flavor compounds in various spirits and their known interactions. It quantifies the strength of interactions between compounds, which can be synergistic (enhancing each other), antagonistic (suppressing each other), or additive. The model identifies key compounds that play central roles in flavor networks, potentially acting as flavor hubs or bridges between different flavor clusters. It can detect clusters of compounds that frequently occur together and contribute to specific flavor notes or profiles.

Using this network structure, the model can predict how changes in the concentration or presence of certain compounds might affect the overall flavor profile. The flavor network analysis model employs graph theory algorithms and machine learning techniques to analyze and make predictions based on these flavor networks. Graph neural networks, community detection algorithms, and link prediction methods work independently and in tandem to predict a complete flavor profile for a distillate. This model is particularly valuable for understanding complex flavor interactions, identifying key flavor compounds, and predicting how changes in production parameters might affect the final flavor profile of a spirit. It can also be used to suggest novel flavor combinations or to understand why certain combinations of ingredients produce particular flavor experiences.

Sensory translation subsystem 840 correlates chemical composition data with human sensory perceptions, translating analytical data into descriptive flavor terms understandable by humans. Its primary function is to bridge the gap between objective chemical data and human sensory perception. The sensory translation subsystem takes the chemical composition data, typically obtained from analytical instruments such as (but not limited to) GC-MS and LC-MS and translates it into descriptive flavor terms that are understandable and meaningful to humans. This translation is crucial because while chemical analysis can provide precise information about the compounds present in a spirit, it doesn't directly convey how these compounds will be perceived by human senses.

Subsystem 840 employs a combination of machine learning models and a comprehensive flavor compound database to achieve this translation. It uses techniques from natural language processing to generate human-readable flavor descriptions from chemical data. This subsystem is critical for making the output of the dynamic flavor prediction and control system useful and actionable for distillers, product developers, and potentially even consumers. It allows for more intuitive understanding and communication about the predicted flavor profiles of spirits.

Historical data analysis unit 850 learns from past production runs to improve future predictions, identifying patterns and trends in successful flavor profiles. This includes information on ingredients, production parameters, process measurements, and the resulting flavor profiles of the finished spirits. The unit uses this historical data to identify patterns, trends, and relationships that can inform future production decisions. The historical data analysis unit uses a combination of statistical techniques and machine learning models to process this data including time series analysis for tracking changes over time, clustering algorithms for grouping similar production runs, and various regression and classification models for predictive tasks. By leveraging past experiences, this unit helps to refine the overall predictive capabilities of the dynamic flavor prediction and control system, contributing to more consistent and higher-quality spirit production over time.

Custom profile development tool 860 allows users to input desired flavor characteristics and suggests production parameters to achieve specified flavor goals. Custom profile development tool leverages the predictive subsystem 810, flavor network analysis subsystem 830, sensory translation subsystem 840, and historical data analysis 850 components of the larger system to generate its suggestions. It serves as a bridge between the complex backend models and the practical needs of distillers and product developers, enabling them to explore new flavor possibilities and fine-tune their products to meet specific flavor targets. The custom development tool 860 is integrated with the adaptive fermentation control system 400, the alcohol removal system 1400, the comprehensive chemical fingerprint system 1200, and a user interface 465. This integration allows for real-time adjustments and optimizations throughout the production workflow, ensuring that the final product achieves the desired flavor profile across different beverage or product categories.

Uncertainty quantification subsystem 870 implements Bayesian neural networks and ensemble methods to provide confidence intervals for flavor predictions. This helps in assessing the reliability of predictions and informs risk management in production decisions.

Flavor evolution trajectory predictor 880 uses the trained RNNs to forecast how flavors will develop over time during fermentation, distillation, and aging. This component helps in optimizing process durations and transition points between production stages. It is trained on historical time-series data of flavor development from past production runs.

Flavor stability modeling unit 890 predicts how flavors will evolve post-production, during aging and storage. It uses a combination of chemical kinetics models and machine learning approaches, trained on long-term studies of spirit aging and storage stability.

Message queue subsystem 899 serves as the central nervous system of the dynamic flavor prediction and control system 800, facilitating real-time data flow and communication between all components. Based on robust stream processing technology, subsystem 899 manages the routing and delivery of data across the entire spirit production process. It receives data streams from various sensors and subsystems, organizes them into topics or channels, and ensures their efficient delivery to the appropriate components for processing. Message queue subsystem 899 interfaces with all other subsystems within system 800, enabling them to publish and consume data as needed. For example, adaptive flavor prediction model 810 uses subsystem 899 to receive real-time production data and publish its predictions and recommendations. Similarly, historical data analysis unit 850 accesses both real-time and historical data streams through subsystem 899. This architecture allows for decoupled, scalable, and fault-tolerant operation, with each component processing data at its own pace while subsystem 899 manages data flow and ensures data integrity. Furthermore, subsystem 899 facilitates integration with external systems 801 such as fermentation system 400, distillation system 600, and aging system 1000, consuming data streams from these systems and publishing control commands and setpoints based on the predictions and recommendations generated by system 800. This design enables seamless data exchange across the entire production process, from fermentation through aging and quality control, allowing for precise optimization of flavor development at every stage.

Subsystem 899 implements real-time feedback mechanisms by continuously processing incoming data streams and generating appropriate responses. For example, it might detect deviations in fermentation parameters and immediately trigger adjustments in the fermentation control system. This real-time feedback functionality is distributed across the system, with each component consuming relevant data streams and publishing responses as needed.

Adaptive flavor prediction model 810 interfaces with subsystem 899 to receive real-time production data and publish its predictions and recommendations. Compound database 820 uses subsystem 899 for updates and queries, ensuring data consistency across the system. The flavor network analysis subsystem 830 and sensory translation subsystem 840 both receive input and publish results via subsystem 899.

Historical data analysis unit 850 accesses both real-time and historical data streams through subsystem 899, enabling it to identify patterns and trends that inform future production decisions. Custom profile development tool 860 interacts with other components through subsystem 899 to access necessary data and publish user-defined profiles.

Flavor evolution trajectory predictor 880 consumes time-series data from subsystem 899 and publishes predictions back to it, while flavor stability modeling unit 890 receives aging- related data and publishes stability predictions. Uncertainty quantification module 870 consumes data from multiple topics in subsystem 899 to perform its analysis and publishes confidence intervals back to the system.

This architecture allows for decoupled, scalable, and fault-tolerant operation, with each component processing data at its own pace while subsystem 899 manages data flow and ensures data integrity. Furthermore, subsystem 899 facilitates integration with external systems such as fermentation system 800, distillation system 600, and aging system 1000, consuming data streams from these systems and publishing control commands and setpoints based on the predictions and recommendations generated by system 800. This design enables seamless data exchange and real-time feedback across the entire production process, from fermentation through aging and quality control, allowing for precise optimization of flavor development at every stage.

Dynamic flavor prediction and control system 800, through its central message queue subsystem 899, seamlessly integrates with other key systems in the spirit production process, forming a comprehensive and intelligent production ecosystem. It interfaces with intelligent control system 300, providing real-time flavor predictions and recommended adjustments to optimize production parameters. The system continuously processes data streams from adaptive fermentation control system 400, analyzing fermentation progress and predicting flavor outcomes. It also interacts closely with advanced distillation system 600, guiding cut points and distillation parameters to achieve desired flavor profiles. Adaptive spirit aging system 1000 receives ongoing input from system 800, adjusting aging conditions based on predicted flavor evolution. For non-alcoholic spirit production, it collaborates with alcohol removal system 1400, ensuring flavor compounds are preserved or reconstructed accurately during dealcoholization. Comprehensive chemical fingerprint system 1200 feeds its detailed chemical analysis into system 800, enhancing the accuracy of flavor predictions and allowing for real-time authentication of flavor profiles. This intricate network of interactions, facilitated by message queue subsystem 899, allows dynamic flavor prediction and control system 800 to maintain a holistic view of the entire production process, from fermentation through aging and potential alcohol removal, enabling precise control and optimization of flavor development at every stage.

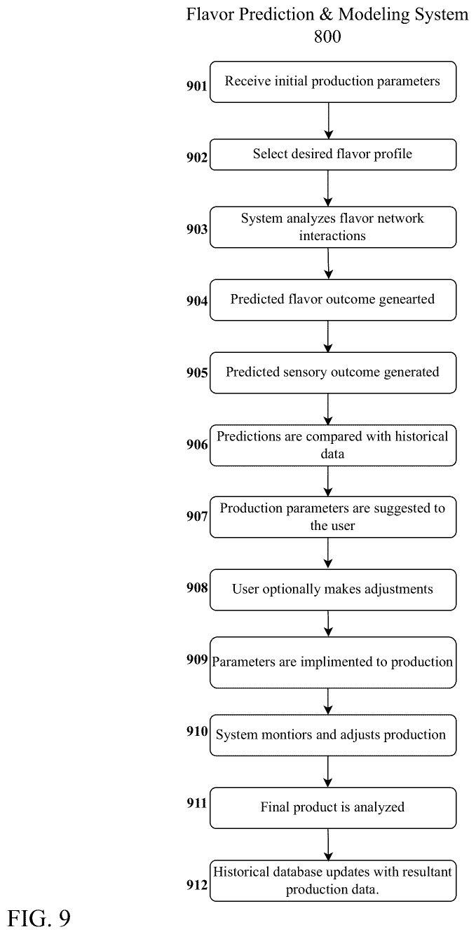

FIG. 9 is a method diagram illustrating the use of dynamic flavor prediction and control system 800. The system first receives the initial production parameters 901. The user selects their desired flavor profile 902. The system analyzes flavor network interactions 903 and a flavor outcome is forecast 904. The flavor prediction 904 is translated into sensory terms 905, and the predictions are compared with historical data 906. A suggested production parameter is then suggested to the user 907. The user may adjust the parameters as needed 908 and may then directly implement the parameters in production 909. Monitoring is done during production and adjustments are made as needed 910. The final product is analyzed 911, and the historical database 850 is updated with the new production data 912.

This sophisticated system enables beverage producers to precisely design and control flavor profiles, innovate new products, and ensure consistency in production. By providing data- driven insights into the complex world of flavor chemistry and perception, dynamic flavor prediction and control system 800 significantly enhances product development processes and helps create beverages tailored to specific consumer preferences or market trends.

### Adaptive Spirit Aging System Overview

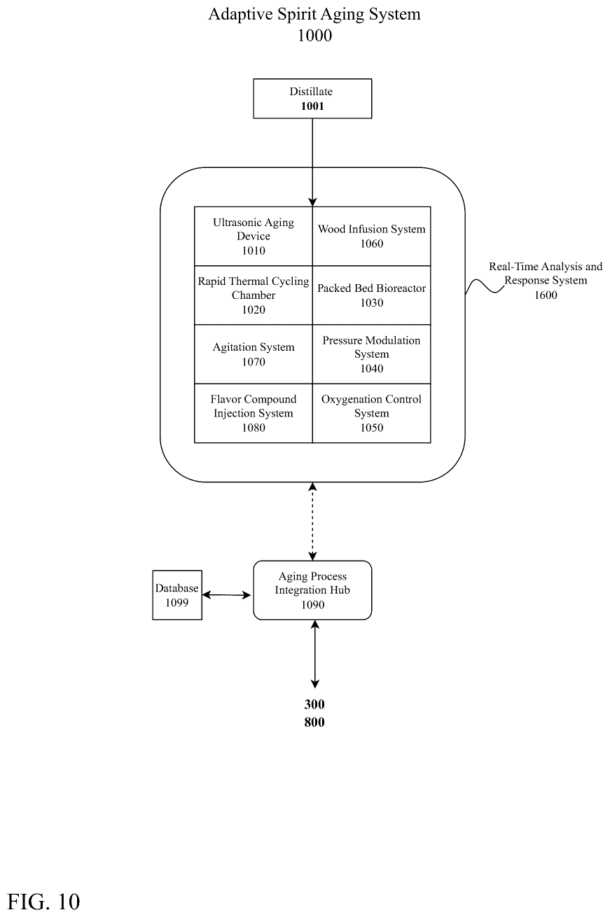

FIG. 10 is a block diagram illustrating an exemplary architecture of adaptive spirit aging system 1000. Adaptive spirit aging system 1000 is designed to optimize and control the maturation process of spirits while maintaining or enhancing quality. The components described may be used in part or in whole to achieve the desired flavor profile from the dynamic flavor prediction and control system 800.

Adaptive spirit aging system 1000 will be described herein with respect to whiskey production, but its capabilities extend to various types of spirits.

As shown, system 1000 comprises several interconnected components: ultrasonic aging device 1010, rapid thermal cycling chamber 1020, packed bed bioreactor 1030, pressure modulation system 1040, oxygenation and micro-oxygenation control system 1050, wood infusion system 1060, agitation system 1070, flavor compound injection system 1080, and aging process integration hub 1090.

Distillate 1001 from advanced distillation system 600 enters adaptive spirit aging system 1000. Ultrasonic aging device 1010 uses ultrasonic waves to accelerate the interaction between the spirit and wood elements. This increases the extraction of wood compounds and speeds up esterification processes, simulating years of aging in a fraction of the time.

Rapid thermal cycling chamber 1020 subjects the spirit to controlled temperature fluctuations. This mimics the seasonal temperature changes that occur during traditional barrel aging, accelerating the maturation process.

Packed bed bioreactor 1030 utilizes a continuous flow system with wood chips or other flavor-imparting materials. This enhances mass transfer rates between the spirit and wood, allowing for precise control of contact time and surface area.

Pressure modulation system 1040 alternates between periods of high pressure and vacuum. This forces the spirit into and out of wood pores, accelerating flavor extraction and chemical reactions.

Oxygenation and micro-oxygenation control system 1050 carefully introduces controlled amounts of oxygen to the aging spirit. This mimics the natural oxygenation that occurs in barrel aging and promotes certain chemical reactions important for flavor development.

Wood infusion system 1060 allows for the introduction of wood chips, staves, or other wood elements. This system can use different wood types or char levels to influence flavor development.

Agitation system 1070 provides controlled movement of the spirit during aging. This ensures even contact with wood elements and promotes uniform aging.

Flavor compound injection system 1080 allows for the precise addition of specific flavor compounds and gases. This can be used to fine-tune the flavor profile during aging, as well as to adjust texture and mouthfeel through the infusion of gases such as nitrogen or carbon dioxide. This versatility enables the system to be adapted for various beverage types, including coffee- based drinks and carbonated spirits.

Aging process integration hub 1090 acts as a central point for data exchange between the adaptive spirit aging system and other systems. It manages the flow of information and control signals to and from other systems, ensuring real-time synchronization of aging processes with overall production goals. The integration hub incorporates a connection to message queue subsystem 899 of dynamic flavor prediction and control system 800. This connection allows for real-time data exchange, including publishing real-time aging data to relevant topics in the message queue and subscribing to flavor prediction and control command topics. Additionally, hub 1090 interfaces with a shared database 899, which stores historical production data, detailed aging parameters and outcomes, and long-term flavor profile evolution data. Integration hub 1090 also features API endpoints for direct communication with intelligent control system 300, enabling the reception of high-level aging strategies and goals, as well as the sending of comprehensive aging status reports.

The system utilizes a continuous learning approach, leveraging the machine learning capabilities of dynamic flavor prediction and control system 800 and intelligent control system 300. Each production run contributes to refining the models across all integrated systems. The feedback loop includes real-time comparison of predicted aging outcomes from system 800 with actual results from system 1000, automated adjustments to aging parameters based on intelligent control system 300 decisions, and storage of comparative data in the shared database for long- term analysis and model improvement. Model validation is performed using held-out test data and expert evaluations to ensure reliability across all integrated systems. The system also incorporates uncertainty quantification methods to manage and communicate the confidence levels of its predictions and decisions, which are shared across systems 300, 800, and 1000 for holistic optimization.

Real-time monitoring system 1600 continuously tracks the aging process using various sensors and analytical instruments. It provides constant updates on the spirit's composition and aging conditions to both the aging process integration hub 1090 and directly to the message queue subsystem 899. This allows for immediate adjustments to achieve the desired flavor profile through the adaptive spirit aging system 1000, real-time flavor prediction updates from system 800, and intelligent control decisions from system 300 based on the latest aging data.

Adaptive spirit aging system 1000 operates as an integral part of the overall intelligent production ecosystem. It receives high-level aging strategies from the intelligent control system 300, implements them using its various components and continuously feeds back results through the aging process integration hub 1090. Dynamic flavor prediction and control system 800 provides real-time flavor evolution predictions, allowing system 1000 to make fine-tuned adjustments to its aging parameters. This tight integration ensures that the aging process is always aligned with overall production goals and desired flavor profiles, while maintaining the flexibility to adapt to unexpected changes or new flavor discoveries during the aging process.

In operation, adaptive spirit aging system 1000 receives distillate from advanced distillation system 600. Aging process integration hub 1090 obtains the target flavor profile from dynamic flavor prediction and control system 800 and initial aging strategies from the intelligent control system 300. Based on these inputs, system 1000 configures the appropriate combination of aging components 1010-1080. As the aging process begins, real-time data from sensors is continuously fed to message queue subsystem 899 and stored in the shared database. System 1000 makes ongoing adjustments to aging parameters based on flavor predictions from system 800 and control decisions from system 300. For example, it might adjust ultrasonic aging device 1010 intensity, modify thermal cycling regime in chamber 1020, or fine-tune oxygenation levels via system 1050. This adaptive approach ensures the aging process remains aligned with the desired flavor profile while optimizing for quality and efficiency. The process continues until the target aging parameters are met, at which point the aged spirit is ready for the next stage of production.

According to another embodiment, adaptive spirit aging system 1000 is configured to accept and process traditionally created spirits. In this scenario, a spirit that has been fermented and distilled using conventional methods is introduced into the system for accelerated aging. System 1000 first analyzes the chemical profile of the traditional spirit using comprehensive chemical fingerprinting system 1200 to establish a baseline. Then, based on the desired final flavor profile, adaptive spirit aging system 1000 employs its various components-such as the ultrasonic aging device 1010, rapid thermal cycling chamber 1020, and other subsystems-to rapidly mature the spirit.

Intelligent control system 300 continuously monitors the aging process, comparing real- time data from the real-time analysis and control system 1600 against predictions from dynamic flavor prediction and control system 800. This allows for precise, iterative adjustments to the aging parameters, optimizing the process for the specific traditional spirit being aged. By applying this accelerated, adaptive aging to traditionally created spirits, the system can dramatically reduce aging time while still achieving complex flavor profiles typically associated with long-term traditional aging methods.

Adaptive spirit aging system 1000 incorporates a dynamic barrel management component that optimizes flavor development through strategic use of different barrel types and sizes. This system can adjust aging parameters in real-time, including temperature, pressure, and critically, the size and type of barrels used. Database 820 stores profiles of various barrel types, including their size, wood type, char level, and characteristic flavor contributions.

As the aging process progresses, the system continuously monitors flavor development using real-time analysis 1600. Based on this data and predictions from flavor modeling system 800, intelligent control system 300 can make decisions to split or combine batches across different barrels to achieve desired flavor profiles. For example, an initial batch might start in a larger barrel, but as specific flavor notes are required, the system could automatically transfer portions to smaller barrels for more rapid flavor exchange. One portion might go to a heavily charred oak barrel for vanilla notes, while another goes to a barrel previously used for spiced rum to impart cinnamon flavors.

System 1000 monitors each sub-batch, optimizing the duration in each barrel type to achieve the desired intensity of specific flavors. Finally, these sub-batches can be recombined and finished in a sherry cask or other finishing barrel to integrate the flavors and add final complexity. This dynamic, responsive approach to barrel aging allows for unprecedented control over the final flavor profile, enabling the creation of highly specific and complex spirits in significantly reduced timeframes.

This embodiment demonstrates the system's flexibility in working with both internally produced and externally sourced spirits, potentially offering significant time and cost savings for traditional distilleries looking to accelerate their aging process without completely overhauling their production methods.

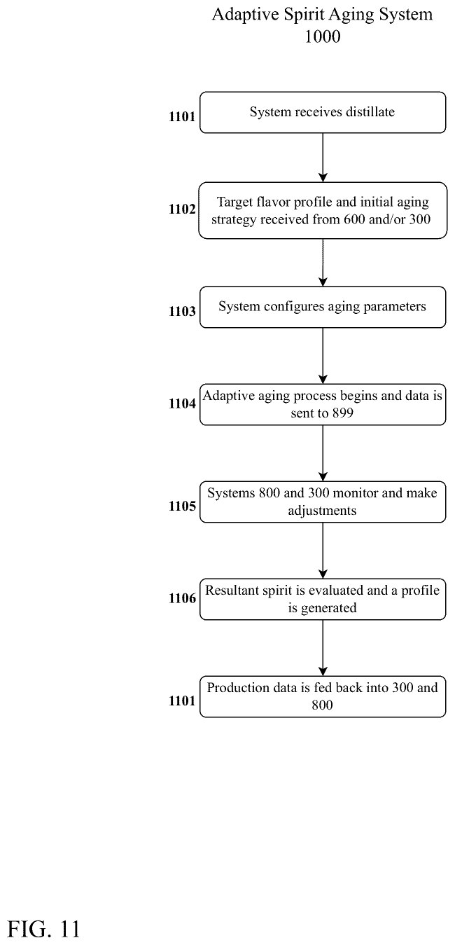

FIG. 11 is a method diagram illustrating the use of adaptive spirit aging system 1000. The distillate from advanced distillation system 600 is received 1101. The target flavor profile and initial aging strategy are received from dynamic flavor prediction and control system 800 and/or intelligent control system 300 1102. The aging parameters are configured based on these inputs 1103. Adaptive aging process begins 1104, with real-time data continuously fed to the message queue subsystem 899. Dynamic flavor prediction system 800 provides ongoing flavor evolution predictions, while intelligent control system 300 suggests adjustments. Based on this feedback, real-time adjustments to the system (pressure, oxygenation, etc.) are made as needed 1105. Throughout the process, data is stored in shared database for long-term analysis. When finished, the resultant spirit is evaluated, a profile is generated 1106, and this information is fed back to systems 300 and 800 for model refinement and future optimization 1107.

Adaptive spirit aging system 1000 is designed with a modular architecture, allowing for easy reconfiguration to produce different types of spirits. It can be scaled from small experimental batches to larger production volumes while maintaining consistency across different batch sizes. The adaptive spirit aging system is also designed to accommodate dynamic changes to barrel sizes across a batch aging system to incorporate different flavor profiles. For example, the aging process may start with a single batch in a single barrel, but then based on sensor feedback the initial batch split into two; one half goes to a “vanilla flavor” barrel and the other half goes to a “cinnamon flavor” barrel. Barrel sizes may vary depending on the strength and speed of the flavor needed to achieve the desired flavor profile. The system would then monitor for the optimal time to impart those flavors, before recombining the batch into a finishing barrel such as a sherry cask.

### Comprehensive Chemical Fingerprint System

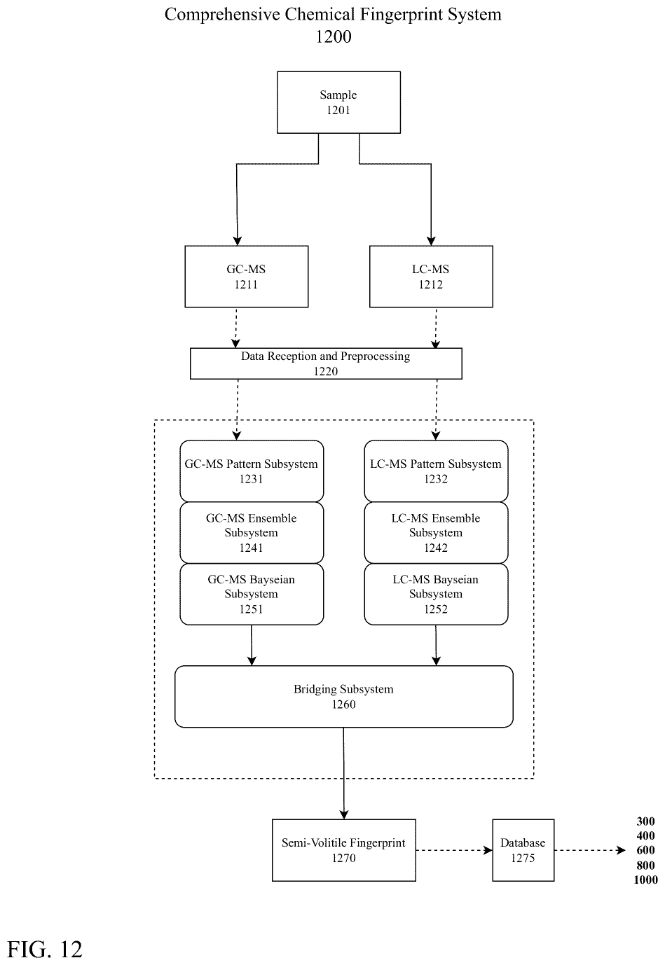

FIG. 12 is a block diagram illustrating an exemplary architecture of comprehensive chemical fingerprint system 1200. Comprehensive chemical fingerprint system 1200 is designed to analyze and characterize the complex chemical profile of spirits, providing a unique “fingerprint” that can be used for quality control, authentication, and flavor profiling.

As shown, system 1200 comprises several interconnected components: analytical instrumentation including Gas Chromatography-Mass Spectrometry (GC-MS) 1211 and Liquid Chromatography-Mass Spectrometry (LC-MS) 1212, data reception and preprocessing subsystem 1220, pattern recognition subsystem 1231/1232, ensemble subsystem 1241/1242, Bayesian subsystem 1251/1252, and bridging system 1260.

Analytical instrumentation may include the use of, for example, GC-MS 1211, LC-MS 1212, or both depending on the specific analysis requirements. GC-MS is particularly effective for analyzing volatile and comprehensive chemical compounds, while LC-MS is better suited for less volatile, larger molecules. Additionally, Nuclear Magnetic Resonance (NMR) spectroscopy, High-Performance Liquid Chromatography (HPLC), Fourier-Transform Infrared Spectroscopy (FTIR) or other instrumentation may be incorporated into the system to meet the molecular identifying needs of system 1200.

Data reception and preprocessing subsystem 1220 receives raw data from analytical instruments 1211 1212 and prepares it for further analysis. This involves noise reduction using Savitzky-Golay filters, baseline correction through asymmetric least squares smoothing, and data normalization techniques such as probabilistic quotient normalization.

Pattern recognition subsystem 1231 1232 analyzes the preprocessed data to identify specific patterns or “signatures” in the chemical profile. This subsystem employs various machine learning techniques including principal component analysis (PCA) for dimensionality reduction, t-distributed stochastic neighbor embedding (t-SNE) for visualization of high- dimensional data, support vector machines for handling non-linear relationships in the expanded chemical space, random forest algorithms for robust classification of diverse chemical signatures, and convolutional neural networks (CNNs) with long short-term memory for feature extraction and classification of spectral patterns. This multi-faceted approach allows the subsystem to recognize and classify patterns across a broad spectrum of chemical compounds, from volatile organics to complex macromolecules, enabling a truly comprehensive chemical fingerprinting capability.

Ensemble subsystem 1241 1242 combines multiple analytical models to improve the overall accuracy and robustness of the fingerprinting process. This includes random forests for handling complex, non-linear relationships in the data, gradient boosting machines for high- performance predictions, and stacking ensembles that leverage the strengths of multiple base models.

Bayesian subsystem 1251 1252 applies probabilistic modeling to the data, allowing for the incorporation of prior knowledge about spirit compositions and the handling of uncertainties in the analysis. This subsystem uses Gaussian process regression for modeling the relationship between chemical composition and sensory attributes, and Bayesian networks for reasoning about the causal relationships between different compounds and their impact on flavor profiles.

When multiple types of instrumentation are employed, the bridging system 1260 comes into play. This system integrates and correlates the data from analytical techniques using canonical correlation analysis (CCA) and partial least squares (PLS) regression. These methods uncover relationships between volatile and non-volatile compounds that contribute to the spirit's overall profile.

The outputs from these subsystems are combined to generate final comprehensive chemical fingerprint 1270. This fingerprint is a comprehensive representation of the spirit's chemical composition, capturing both the presence and quantities of various compounds as well as their interrelationships. Fingerprint is stored in database 1275 accessible to outside systems such as 300 or 600.

Comprehensive chemical fingerprint system 1200 is tightly integrated with other key systems in the patent. It provides real-time data to adaptive spirit aging system 1000, allowing for precise adjustments to the aging process based on the evolving chemical profile of the spirit. System 1200 also feeds data to dynamic flavor prediction and control system 800, enhancing the accuracy of flavor predictions and enabling more precise control over the final product.

The generated fingerprints are stored in high-dimensional database 1275 using locality- sensitive hashing for efficient similarity searches. This allows for rapid comparison of new profiles against known standards for authentication and quality control. System employs a hierarchical clustering algorithm to organize fingerprints based on similarity, facilitating the identification of trends and anomalies across different batches or production runs.

To handle different types of spirits and adapt to new or unusual profiles, the system incorporates a transfer learning approach. This allows knowledge gained from analyzing one type of spirit to be applied to others, improving performance even with limited data for new spirit types. The system also includes an anomaly detection module based on isolation forests, which can identify and flag unusual chemical profiles for further investigation.

In operation, comprehensive chemical fingerprint system 1200 begins by analyzing a spirit sample using its analytical instrumentation, employing for example both GC-MS 1211 and LC-MS 1212 techniques for comprehensive chemical profiling. The raw data from these instruments is then fed into data reception and preprocessing subsystem 1220, which cleans and normalizes data using advanced filtering and smoothing algorithms. Next, pattern recognition subsystem 1231/1232 analyzes this preprocessed data to identify specific chemical signatures, utilizing machine learning techniques such as principal component analysis and convolutional neural networks. Ensemble subsystem 1241/1242 then combines multiple analytical models to improve the overall accuracy and robustness of the analysis. Concurrently, Bayesian subsystem 1251/1252 applies probabilistic modeling to the data, incorporating prior knowledge about spirit compositions and handling uncertainties. When both GC-MS and LC-MS data are available, bridging system 1260 integrates and correlates this information to provide a more comprehensive analysis. The final output is a detailed comprehensive chemical fingerprint 1270 of the spirit, which is then compared against database 1275 of known profiles for quality assurance, authenticity verification, or flavor profiling. This fingerprint can also be used to inform other systems, such as adaptive spirit aging system 1000 or dynamic flavor prediction and control system 800, allowing for real-time adjustments to the production process.

Comprehensive chemical fingerprinting system's 1200 broad analysis enhances quality control and authenticity verification in several practical ways. In quality control, system 1200 might detect a batch of whiskey with slightly elevated levels of certain esters, indicating a minor deviation in the fermentation process. This early detection allows for immediate corrective action, ensuring the final product maintains the distillery's established flavor profile. Similarly, for a gin producer, the system could identify subtle variations in botanical extracts between batches, allowing for precise adjustments to maintain consistent flavor.

For authenticity verification, consider a case of rare, aged rum. The system's 1200 detailed analysis would reveal not only the expected congeners from aging, but also trace compounds unique to the distillery's water source and fermentation environment. This comprehensive chemical signature makes it extremely difficult for counterfeiters to replicate the product accurately. In another scenario, a premium vodka brand could use the system to verify the authenticity of its products in different markets. The detailed chemical profile would reflect the specific grain mix and filtration processes unique to that brand, readily distinguishing genuine products from sophisticated imitations.

These capabilities ensure that spirits producers can maintain high standards of quality and protect the integrity of their brands in an increasingly complex global market.

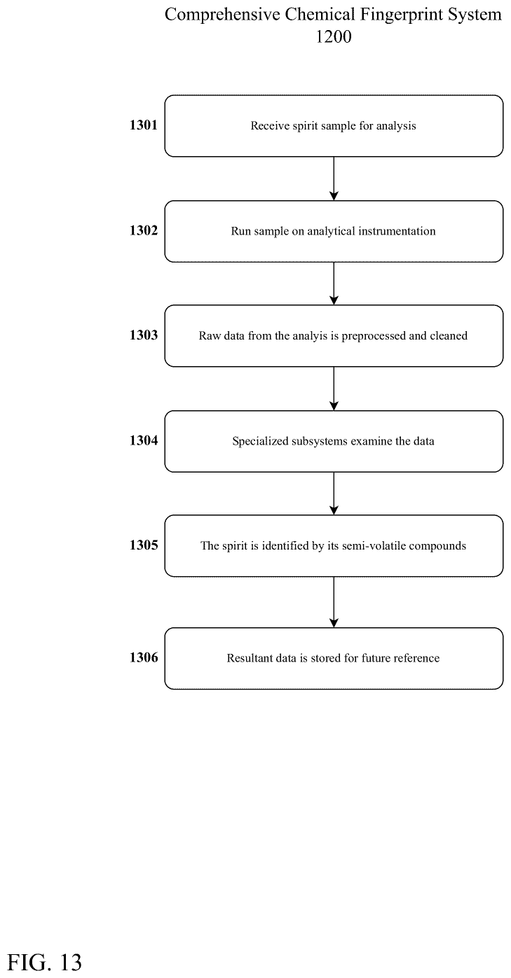

FIG. 13 is a method diagram illustrating the use of comprehensive chemical fingerprint system 1200. A spirit sample is received for analysis 1301 and run on analytical instrumentation 1302. The raw data from the analysis is preprocessed and cleaned 1303 and sent to specialized subsystems to examine the resultant data 1304. The spirit is identified by its comprehensive chemical components 1305 and the resultant data is stored in database 1275 for future use and reference 1306.

Comprehensive chemical fingerprint system 1200 can be used at various stages of the production process, from assessing raw ingredients to quality control of the final product. The generated fingerprints can be compared against a database of known profiles for authentication purposes, used to track the evolution of a spirit during aging, or employed to ensure consistency across different batches. System's 1200 adaptive capabilities ensure that it remains effective and accurate even as production methods evolve or new types of spirits are introduced.

### Alcohol Removal System

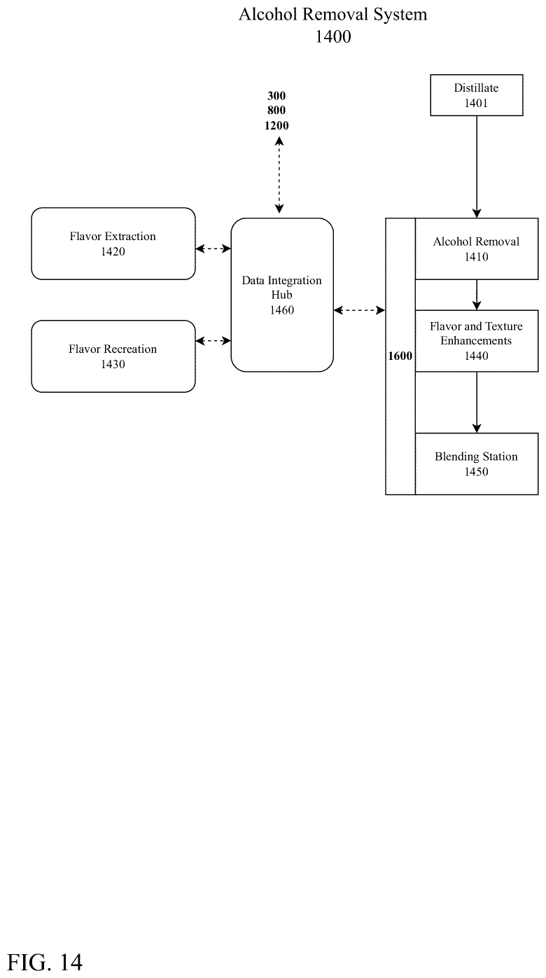

FIG. 14 is a block diagram illustrating an exemplary architecture of alcohol removal system 1400. Alcohol removal system 1400 is designed to create complex, flavorful non- alcoholic beverages that mimic the taste and experience of traditional spirits.

As shown, system 1400 comprises several interconnected components: alcohol removal unit 1410, flavor extraction system 1420, flavor reconstruction subsystem 1430, texture enhancement system 1440, blending station 1450, and data integration hub 1460.

Alcohol removal unit 1410 is the core component of the system, utilizing advanced techniques such as vacuum distillation and/or reverse osmosis to remove alcohol from the spirit. This unit is designed to preserve delicate flavors and aromas during the de-alcoholization process, which is crucial for maintaining the spirit's character in the non-alcoholic version.

Flavor extraction system 1420 employs sophisticated extraction techniques to isolate key flavor compounds from the original alcoholic spirit or from raw ingredients. This involves methods such as supercritical fluid extraction or molecular distillation for precise flavor isolation. The goal is to capture the essential flavor elements that give the original spirit its distinctive taste profile. Comprehensive chemical fingerprint system 1200 is employed to create a detailed chemical profile of the original alcoholic base, which serves as a target for flavor reconstruction.

Flavor reconstruction subsystem 1430 is responsible for recombining the extracted flavor compounds to recreate complex flavor profiles. This subsystem works in close conjunction with dynamic flavor prediction and control system 800, utilizing advanced machine learning algorithms to achieve desired taste outcomes that closely mimic the original alcoholic beverage.

Flavor reconstruction subsystem 1430 employs a combination of deep neural networks and Gaussian process regression models. Deep neural networks are trained on extensive datasets of chemical profiles and corresponding sensory evaluations, allowing them to learn the complex relationships between chemical composition and perceived flavor. These networks use transfer learning techniques, leveraging knowledge gained from modeling alcoholic spirits to improve performance on non-alcoholic alternatives. Gaussian process regression models are used to handle uncertainty in the flavor reconstruction process, providing confidence intervals for predicted flavor outcomes. These models are trained using historical data from successful non- alcoholic spirit productions, continually updated with new data to improve accuracy over time.

The training process for these models is a comprehensive, multi-stage approach designed to ensure optimal performance and adaptability. It begins with initial training on a large dataset of spirit chemical profiles and corresponding sensory data, establishing a robust foundation for flavor prediction. This is followed by fine-tuning on specific spirit types using transfer learning, allowing the models to specialize in particular flavor profiles. The system then employs continuous learning through online updates as new production data becomes available, enabling real-time improvements in accuracy. To maintain overall performance and prevent catastrophic forgetting, periodic retraining is conducted on the entire dataset. This iterative process ensures that the models remain current, accurate, and capable of handling the complex task of flavor reconstruction across a wide range of non-alcoholic spirits.

Texture enhancement system 1440 adds compounds to mimic the mouthfeel and body of alcoholic beverages. This may include the addition of glycerin or other non-alcoholic bases to replicate the viscosity and ‘warmth’ typically associated with alcoholic spirits. The system uses a reinforcement learning algorithm to optimize the combination and quantities of texture enhancers, with the reward function based on sensory evaluation scores.

Blending station 1450 precisely mixes various flavor components, texture enhancers, and base liquids. It utilizes a continuous flow system for accurate proportioning, ensuring consistency in the final product. The blending process is guided by a multi-objective optimization algorithm that balances flavor fidelity, texture, and production efficiency.

Data integration hub 1460 serves as the central point for data exchange and model coordination across the alcohol removal system and other systems. It interfaces with the real- time analysis and response system 1600 to receive continuous updates on the dealcoholization process, flavor extraction, and blending operations. This data is used to adjust model predictions and control parameters in real-time.

Hub 1460 also communicates with the dynamic flavor prediction and control system 800, sharing data on flavor compound concentrations and sensory outcomes. This bidirectional flow of information allows both systems to improve their predictive accuracy over time.

Importantly, data integration hub 1460 directly interfaces with intelligent control system 300. It receives high-level directives and optimization goals from intelligent control system 300 based on its analysis of the entire production process. In turn, hub 1460 sends real-time data on the alcohol removal process, including dealcoholization efficiency, flavor retention, and blending outcomes, back to intelligent control system 300. This allows for dynamic adjustments to the alcohol removal process based on broader production goals and constraints.

In operation, alcohol removal system 1400 functions as an integrated, intelligent process for creating non-alcoholic spirits with complex flavor profiles. The system begins with either an alcoholic spirit or a specifically crafted blend designed for dealcoholization. Alcohol removal unit 1410 employs advanced techniques such as vacuum distillation or reverse osmosis to remove alcohol while minimizing flavor loss. Concurrently, flavor extraction system 1420 works to isolate and preserve key flavor compounds using methods like supercritical fluid extraction or molecular distillation.

The extracted flavors are then carefully reconstructed in flavor reconstruction subsystem 1430, which utilizes deep neural networks and Gaussian process regression models to recreate complex flavor profiles. Texture enhancement system 1440 adds compounds to mimic the mouthfeel of alcoholic beverages, while blending station 1450 precisely combines all components. Throughout this process, data integration hub 1460 coordinates information flow between system components and interfaces with intelligent control system 300 and dynamic flavor prediction and control system 800, ensuring that the final non-alcoholic product closely matches the target flavor profile and meets broader production goals.

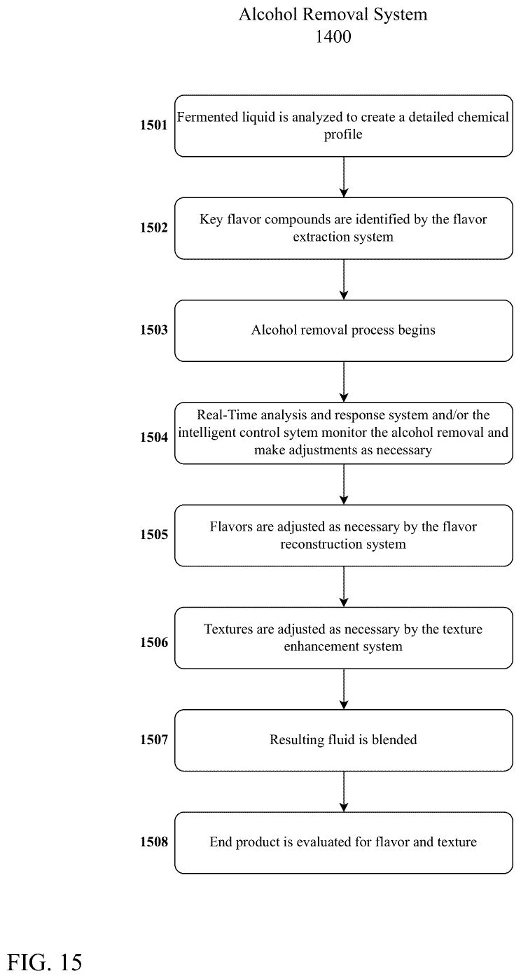

FIG. 15 is a method diagram illustrating the use of alcohol removal system 1400. The fermented liquid to undergo the process is first analyzed to create a detailed chemical profile 1501 and key flavor compounds are identified 1502. The alcohol removal process begins 1503, under the guidance of the real-time monitoring system 1600 and/or the intelligent control system 300. Flavors are adjusted as necessary 1505 as is texture 1506. The resultant liquid is blended 1507 and the end product is finalized for flavor and texture 1508. Throughout this process, the machine learning models continuously update their predictions based on real-time data, optimizing the production process and final product quality.

This sophisticated alcohol removal system 1400 enables the production of high-quality, non-alcoholic alternatives that closely mimic the complexity and experience of traditional spirits. By combining advanced extraction techniques, adaptive flavor reconstruction, and careful formulation, this system can produce non-alcoholic beverages that satisfy the growing demand for alcohol-free options without compromising on taste or complexity. The integration of machine learning throughout the process ensures continuous improvement in product quality and production efficiency.

### Real-Time Analysis and Response System

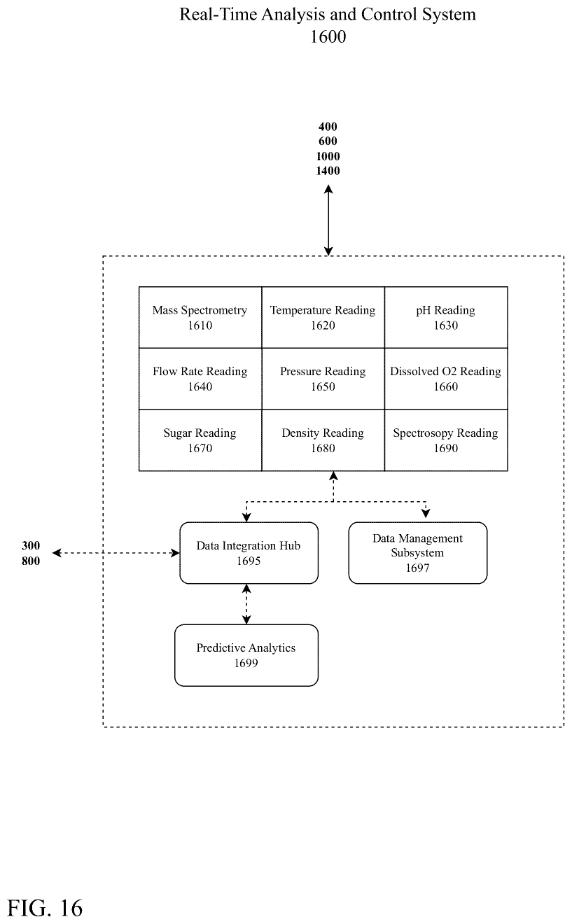

FIG. 16 is a block diagram illustrating an exemplary architecture of real-time analysis and control system 1600. System 1600 is designed to provide continuous, comprehensive data collection, immediate analysis, and real-time process control across various aspects of the spirit production process, enabling unprecedented levels of control and optimization.

As shown, system 1600 comprises several interconnected components that can be configured based on specific production needs: mass spectrometry unit 1610, temperature monitoring unit 1620, pH monitoring unit 1630, flow rate monitoring unit 1640, pressure monitoring unit 1650, dissolved oxygen monitoring unit 1660, sugar monitoring unit 1670, density monitoring unit 1680, spectroscopy unit 1690, central data integration hub 1695, data management subsystem 1697, and adaptive predictive modeling subsystem 1699.

Mass spectrometry unit 1610 may include Gas Chromatography-Mass Spectrometry (GC-MS) and/or Liquid Chromatography-Mass Spectrometry (LC-MS) capabilities, depending on the specific analysis requirements. This unit provides detailed, real-time analysis of the chemical composition of the spirit throughout the production process, from fermentation through distillation and aging.

Temperature monitoring unit 1620 uses a network of high-precision thermocouples placed at various points in the production line to ensure optimal temperature control for each process stage. This is crucial for maintaining consistency in fermentation, distillation, and aging processes.

PH monitoring unit 1630 continuously tracks acidity levels throughout production using ion-selective field-effect transistor (ISFET) sensors. This is particularly important during fermentation and in ensuring product consistency.

Flow rate monitoring unit 1640 employs ultrasonic flow meters to measure the flow of spirits and other liquids through the system, ensuring precise control of blending and aging processes.

Pressure monitoring unit 1650 uses piezoresistive pressure sensors to track pressure in distillation columns, reactors, and other vessels, which is essential for process control and safety.

Dissolved oxygen monitoring unit 1660 utilizes optical oxygen sensors based on fluorescence quenching to measure oxygen levels, particularly important during fermentation and aging. It helps control oxidation processes that can significantly impact flavor development.

Sugar monitoring unit 1670 employs near-infrared (NIR) spectroscopy to track sugar content throughout the fermentation process, providing crucial data on fermentation progress and efficiency.

Density monitoring unit 1680 uses vibrating tube density meters to continuously measure the density of the spirit, helping to monitor alcohol content and fermentation progress.

Spectroscopy unit 1690 includes both Fourier-transform infrared (FTIR) and UV-visible spectroscopy for rapid, non-destructive analysis of various spirit qualities, including color, clarity, and specific chemical markers.

All active monitoring units feed data into central data integration hub 1695, which collects, centralizes, and performs initial processing of information from all monitoring devices. This hub ensures that all data is time-stamped, properly formatted for analysis, and securely stored. It also implements immediate, low-level process control adjustments based on predefined parameters. Central data integration hub 1695 serves as the primary interface with other key systems, including intelligent control system 300 and dynamic flavor prediction and control system 800. It sends analyzed data and immediate process adjustment reports to these systems, and receives high-level production strategies, target parameters, and flavor profile targets. This bidirectional communication enables seamless integration between real-time monitoring, higher- level decision-making processes, and flavor control operations.

Data management subsystem 1697 is responsible for the efficient storage, retrieval, and management of the vast amounts of data generated by the real-time analysis and response system 1600. This subsystem employs a distributed, scalable database architecture using technologies such as Apache Cassandra for time-series data storage and Apache Kafka for real-time data streaming.

Key features of the data management subsystem 1697 include high-speed data ingestion capabilities to handle the continuous influx of sensor data from all monitoring units, efficient data compression and storage optimization techniques to manage large volumes of historical data, and advanced data partitioning and indexing strategies for rapid data retrieval and query performance. The subsystem also provides automated data lifecycle management, including data archiving and purging policies to balance storage costs with data availability needs, real-time and batch data processing capabilities to support both immediate analysis and long-term trend identification, robust data backup and recovery mechanisms to ensure data integrity and system reliability, and fine-grained access control and encryption to maintain data security and comply with relevant regulations.

Data management subsystem 1697 interfaces closely with central data integration hub 1695 and adaptive predictive modeling subsystem 1699, ensuring that all components of real- time analysis and response system 1600 have access to the data they need, when they need it. This subsystem also facilitates data sharing with other systems in the overall spirit production process, such as dynamic flavor prediction and control system 400 and comprehensive chemical fingerprint system 1200, enabling comprehensive data-driven decision making across the entire production pipeline.

Adaptive predictive modeling subsystem 1699 takes integrated data and applies advanced machine learning algorithms to analyze real-time data streams. It identifies patterns and trends, predicts potential issues or quality deviations before they occur, and suggests proactive adjustments to maintain optimal production conditions. This subsystem continuously learns from historical data to improve its predictive accuracy over time.

Adaptive predictive modeling subsystem 1699 uses an ensemble of machine learning techniques such as Long Short-Term Memory (LSTM) networks for capturing complex temporal dependencies in the production process, Random Forest algorithms for robust prediction of multiple output variables, Gaussian Process Regression for uncertainty quantification in predictions, and Reinforcement Learning agents for optimizing control parameters over time, learning from the outcomes of different production runs.

Real-time analysis and control system 1600 interfaces closely with other components of the integrated system, particularly the intelligent control system 300 and the adaptive spirit aging system 1000. It provides analyzed data and immediate process adjustment reports necessary for these systems to make informed decisions and high-level adjustments to the production process in real-time. In turn, it receives high-level production strategies and target parameters from these systems.

In operation, system 1600 functions as a continuous, adaptive monitoring and control loop. The active monitoring units constantly collect real-time data from various stages of production, which is then received, time-stamped, and initially validated by the central data integration hub 1695. This hub conducts rapid analysis of incoming data to detect any critical changes or anomalies requiring immediate attention. Based on predefined parameters and this immediate analysis, the system implements low-level adjustments to maintain optimal conditions, such as tweaking fermentation temperature or distillation pressure in real-time. Concurrently, the data management subsystem 1697 preprocesses the collected data, applying necessary cleaning and normalization techniques. Adaptive predictive modeling subsystem 1699 then analyzes this processed data to identify patterns, predict potential issues, and suggest proactive adjustments. System 1600 streams relevant processed data and analysis results to other systems for higher-level decision making, while also incorporating feedback from these systems to adjust its monitoring priorities and control parameters. This cycle operates continuously, with the system adapting its focus based on the current production stage. For instance, during fermentation, it prioritizes temperature, pH, sugar content, and dissolved oxygen monitoring, while during distillation, it shifts focus to temperature, pressure, flow rates, and chemical composition analysis. Through this dynamic, responsive process, system 1600 ensures optimal conditions are maintained throughout spirit production.

This comprehensive, real-time monitoring and control allows for proactive management of the production process, ensuring consistent quality and enabling rapid response to any issues that may arise. It also provides valuable data for dynamic flavor prediction and control system 800 and comprehensive chemical fingerprint system 1200, contributing to the overall efficiency and quality of the spirit production process.

Real-time analysis and control system 1600 represents a significant advancement in spirit production technology, providing a level of insight, control, and immediate responsiveness previously unattainable in traditional distillation processes. Its modular design allows for customization based on specific production needs, ensuring optimal performance across various types of spirit production.

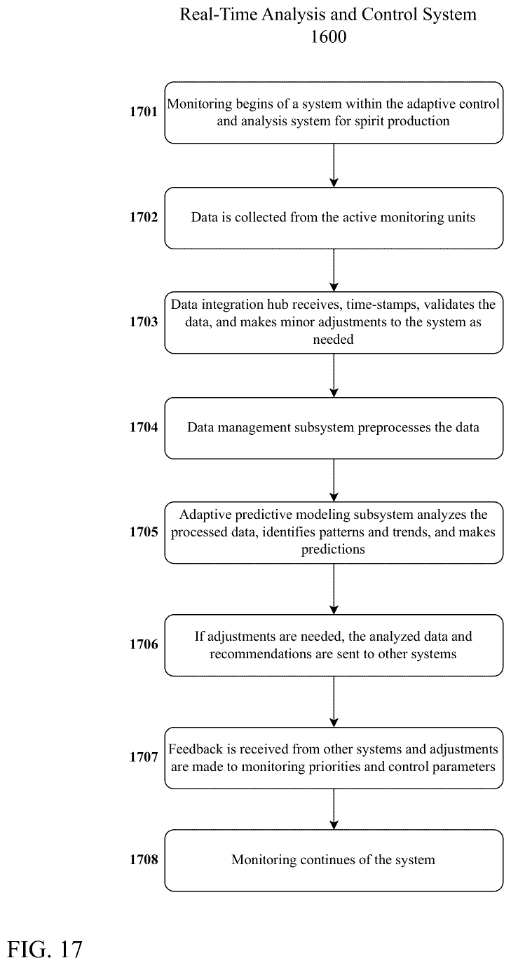

FIG. 17 is a method diagram illustrating the operation of real-time analysis and control system 1600. A system within the adaptive control and analysis system for spirit production such as 400 or 1000 begins being monitored 1701. Continuous data collection from active monitoring units 1610-1690 1702. This data is received by central data integration hub 1695, where it is time-stamped and initially validated 1703. Hub 1695 performs immediate analysis to detect critical changes or anomalies, implementing low-level adjustments in real-time if necessary. Concurrently, data management subsystem 1697 preprocesses, cleans, and stores data 1704. Adaptive predictive modeling subsystem 1699 then analyzes this processed data, identifying patterns, trends, and making predictions 1705. The analyzed data and any recommendations are then distributed to other systems 1706, such as intelligent control system 300 and dynamic flavor prediction and control system 800. Feedback, including updated strategies and parameters, is received from these systems 1707. This process operates as a continuous loop 1708, constantly adapting to the current production stage and requirements, ensuring optimal spirit production conditions are maintained throughout.

### Exemplary Computing Environment

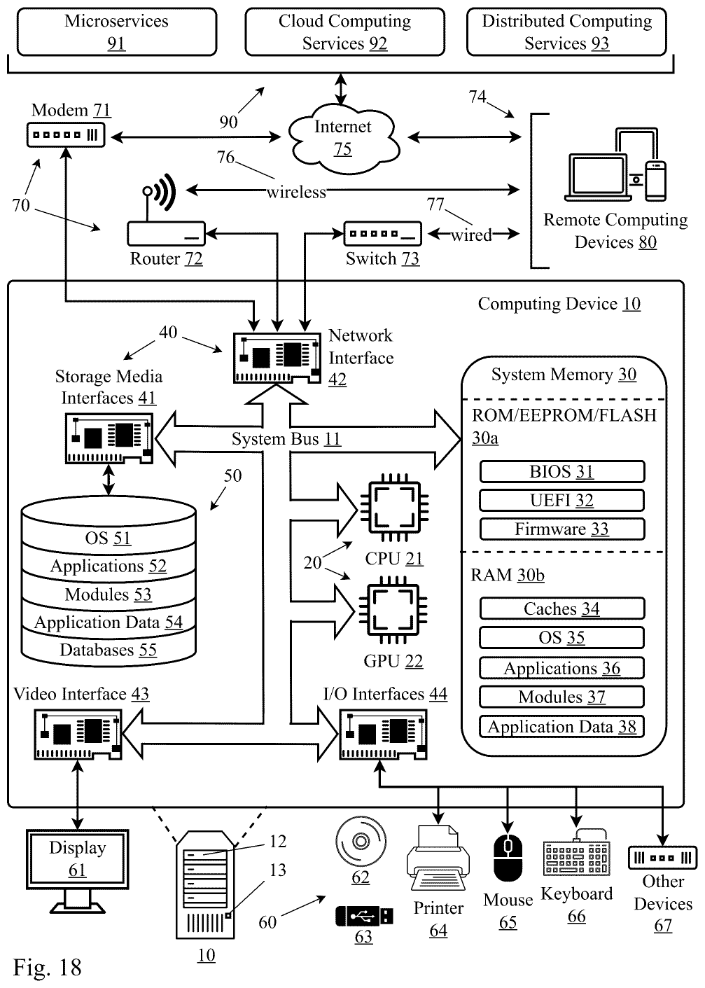

FIG. 18 illustrates an exemplary computing environment on which an embodiment described herein may be implemented, in full or in part. This exemplary computing environment describes computer-related components and processes supporting enabling disclosure of computer-implemented embodiments. Inclusion in this exemplary computing environment of well-known processes and computer components, if any, is not a suggestion or admission that any embodiment is no more than an aggregation of such processes or components. Rather, implementation of an embodiment using processes and components described in this exemplary computing environment will involve programming or configuration of such processes and components resulting in a machine specially programmed or configured for such implementation. The exemplary computing environment described herein is only one example of such an environment and other configurations of the components and processes are possible, including other relationships between and among components, and/or absence of some processes or components described. Further, the exemplary computing environment described herein is not intended to suggest any limitation as to the scope of use or functionality of any embodiment implemented, in whole or in part, on components or processes described herein.

The exemplary computing environment described herein comprises a computing device 10 (further comprising a system bus 11, one or more processors 20, a system memory 30, one or more interfaces 40, one or more non-volatile data storage devices 50), external peripherals and accessories 60, external communication devices 70, remote computing devices 80, and cloud- based services 90.

System bus 11 couples the various system components, coordinating operation of and data transmission between those various system components. System bus 11 represents one or more of any type or combination of types of wired or wireless bus structures including, but not limited to, memory busses or memory controllers, point-to-point connections, switching fabrics, peripheral busses, accelerated graphics ports, and local busses using any of a variety of bus architectures. By way of example, such architectures include, but are not limited to, Industry Standard Architecture (ISA) busses, Micro Channel Architecture (MCA) busses, Enhanced ISA (EISA) busses, Video Electronics Standards Association (VESA) local busses, a Peripheral Component Interconnects (PCI) busses also known as a Mezzanine busses, or any selection of, or combination of, such busses. Depending on the specific physical implementation, one or more of the processors 20, system memory 30 and other components of the computing device 10 can be physically co-located or integrated into a single physical component, such as on a single chip. In such a case, some or all of system bus 11 can be electrical pathways within a single chip structure.

Computing device may further comprise externally-accessible data input and storage devices 12 such as compact disc read-only memory (CD-ROM) drives, digital versatile discs (DVD), or other optical disc storage for reading and/or writing optical discs 62; magnetic cassettes, magnetic tape, magnetic disk storage, or other magnetic storage devices; or any other medium which can be used to store the desired content and which can be accessed by the computing device 10. Computing device may further comprise externally-accessible data ports or connections 12 such as serial ports, parallel ports, universal serial bus (USB) ports, and infrared ports and/or transmitter/receivers. Computing device may further comprise hardware for wireless communication with external devices such as IEEE 1394 (“Firewire”) interfaces, IEEE 802.11 wireless interfaces, BLUETOOTH® wireless interfaces, and so forth. Such ports and interfaces may be used to connect any number of external peripherals and accessories 60 such as visual displays, monitors, and touch-sensitive screens 61, USB solid state memory data storage drives (commonly known as “flash drives” or “thumb drives”) 63, printers 64, pointers and manipulators such as mice 65, keyboards 66, and other devices 67 such as joysticks and gaming pads, touchpads, additional displays and monitors, and external hard drives (whether solid state or disc-based), microphones, speakers, cameras, and optical scanners.

Processors 20 are logic circuitry capable of receiving programming instructions and processing (or executing) those instructions to perform computer operations such as retrieving data, storing data, and performing mathematical calculations. Processors 20 are not limited by the materials from which they are formed or the processing mechanisms employed therein, but are typically comprised of semiconductor materials into which many transistors are formed together into logic gates on a chip (i.e., an integrated circuit or IC). The term processor includes any device capable of receiving and processing instructions including, but not limited to, processors operating on the basis of quantum computing, optical computing, mechanical computing (e.g., using nanotechnology entities to transfer data), and so forth. Depending on configuration, computing device 10 may comprise more than one processor. For example, computing device 10 may comprise one or more central processing units (CPUs) 21, each of which itself has multiple processors or multiple processing cores, each capable of independently or semi-independently processing programming instructions based on technologies like complex instruction set computer (CISC) or reduced instruction set computer (RISC). Further, computing device 10 may comprise one or more specialized processors such as a graphics processing unit (GPU) 22 configured to accelerate processing of computer graphics and images via a large array of specialized processing cores arranged in parallel. Further computing device 10 may be comprised of one or more specialized processes such as Intelligent Processing Units, field- programmable gate arrays or application-specific integrated circuits for specific tasks or types of tasks. The term processor may further include: neural processing units (NPUs) or neural computing units optimized for machine learning and artificial intelligence workloads using specialized architectures and data paths; tensor processing units (TPUs) designed to efficiently perform matrix multiplication and convolution operations used heavily in neural networks and deep learning applications; application-specific integrated circuits (ASICs) implementing custom logic for domain-specific tasks; application-specific instruction set processors (ASIPs) with instruction sets tailored for particular applications; field-programmable gate arrays (FPGAs) providing reconfigurable logic fabric that can be customized for specific processing tasks; processors operating on emerging computing paradigms such as quantum computing, optical computing, mechanical computing (e.g., using nanotechnology entities to transfer data), and so forth. Depending on configuration, computing device 10 may comprise one or more of any of the above types of processors in order to efficiently handle a variety of general purpose and specialized computing tasks. The specific processor configuration may be selected based on performance, power, cost, or other design constraints relevant to the intended application of computing device 10.

System memory 30 is processor-accessible data storage in the form of volatile and/or nonvolatile memory. System memory 30 may be either or both of two types: non-volatile memory and volatile memory. Non-volatile memory 30a is not erased when power to the memory is removed, and includes memory types such as read only memory (ROM), electronically-erasable programmable memory (EEPROM), and rewritable solid state memory (commonly known as “flash memory”). Non-volatile memory 30a is typically used for long-term storage of a basic input/output system (BIOS) 31, containing the basic instructions, typically loaded during computer startup, for transfer of information between components within computing device, or a unified extensible firmware interface (UEFI), which is a modern replacement for BIOS that supports larger hard drives, faster boot times, more security features, and provides native support for graphics and mouse cursors. Non-volatile memory 30a may also be used to store firmware comprising a complete operating system 35 and applications 36 for operating computer-controlled devices. The firmware approach is often used for purpose-specific computer-controlled devices such as appliances and Internet-of-Things (IoT) devices where processing power and data storage space is limited. Volatile memory 30b is erased when power to the memory is removed and is typically used for short-term storage of data for processing. Volatile memory 30b includes memory types such as random-access memory (RAM), and is normally the primary operating memory into which the operating system 35, applications 36, program modules 37, and application data 38 are loaded for execution by processors 20. Volatile memory 30b is generally faster than non-volatile memory 30a due to its electrical characteristics and is directly accessible to processors 20 for processing of instructions and data storage and retrieval. Volatile memory 30b may comprise one or more smaller cache memories which operate at a higher clock speed and are typically placed on the same IC as the processors to improve performance.

There are several types of computer memory, each with its own characteristics and use cases. System memory 30 may be configured in one or more of the several types described herein, including high bandwidth memory (HBM) and advanced packaging technologies like chip-on-wafer-on-substrate (CoWoS). Static random access memory (SRAM) provides fast, low-latency memory used for cache memory in processors, but is more expensive and consumes more power compared to dynamic random access memory (DRAM). SRAM retains data as long as power is supplied. DRAM is the main memory in most computer systems and is slower than SRAM but cheaper and more dense. DRAM requires periodic refresh to retain data. NAND flash is a type of non-volatile memory used for storage in solid state drives (SSDs) and mobile devices and provides high density and lower cost per bit compared to DRAM with the trade-off of slower write speeds and limited write endurance. HBM is an emerging memory technology that provides high bandwidth and low power consumption which stacks multiple DRAM dies vertically, connected by through-silicon vias (TSVs). HBM offers much higher bandwidth (up to 1 TB/s) compared to traditional DRAM and may be used in high-performance graphics cards, AI accelerators, and edge computing devices. Advanced packaging and CoWoS are technologies that enable the integration of multiple chips or dies into a single package. CoWoS is a 2.5D packaging technology that interconnects multiple dies side-by-side on a silicon interposer and allows for higher bandwidth, lower latency, and reduced power consumption compared to traditional PCB-based packaging. This technology enables the integration of heterogeneous dies (e.g., CPU, GPU, HBM) in a single package and may be used in high-performance computing, AI accelerators, and edge computing devices.

Interfaces 40 may include, but are not limited to, storage media interfaces 41, network interfaces 42, display interfaces 43, and input/output interfaces 44. Storage media interface 41 provides the necessary hardware interface for loading data from non-volatile data storage devices 50 into system memory 30 and storage data from system memory 30 to non- volatile data storage device 50. Network interface 42 provides the necessary hardware interface for computing device 10 to communicate with remote computing devices 80 and cloud-based services 90 via one or more external communication devices 70. Display interface 43 allows for connection of displays 61, monitors, touchscreens, and other visual input/output devices. Display interface 43 may include a graphics card for processing graphics-intensive calculations and for handling demanding display requirements. Typically, a graphics card includes a graphics processing unit (GPU) and video RAM (VRAM) to accelerate display of graphics. In some high- performance computing systems, multiple GPUs may be connected using NVLink bridges, which provide high-bandwidth, low-latency interconnects between GPUs. NVLink bridges enable faster data transfer between GPUs, allowing for more efficient parallel processing and improved performance in applications such as machine learning, scientific simulations, and graphics rendering. One or more input/output (I/O) interfaces 44 provide the necessary support for communications between computing device 10 and any external peripherals and accessories 60. For wireless communications, the necessary radio-frequency hardware and firmware may be connected to I/O interface 44 or may be integrated into I/O interface 44. Network interface 42 may support various communication standards and protocols, such as Ethernet and Small Form-Factor Pluggable (SFP). Ethernet is a widely used wired networking technology that enables local area network (LAN) communication. Ethernet interfaces typically use RJ45 connectors and support data rates ranging from 10 Mbps to 100 Gbps, with common speeds being 100 Mbps, 1 Gbps, 10 Gbps, 25 Gbps, 40 Gbps, and 100 Gbps. Ethernet is known for its reliability, low latency, and cost-effectiveness, making it a popular choice for home, office, and data center networks. SFP is a compact, hot-pluggable transceiver used for both telecommunication and data communications applications. SFP interfaces provide a modular and flexible solution for connecting network devices, such as switches and routers, to fiber optic or copper networking cables. SFP transceivers support various data rates, ranging from 100 Mbps to 100 Gbps, and can be easily replaced or upgraded without the need to replace the entire network interface card. This modularity allows for network scalability and adaptability to different network requirements and fiber types, such as single-mode or multi-mode fiber.

Non-volatile data storage devices 50 are typically used for long-term storage of data. Data on non-volatile data storage devices 50 is not erased when power to the non-volatile data storage devices 50 is removed. Non-volatile data storage devices 50 may be implemented using any technology for non-volatile storage of content including, but not limited to, CD-ROM drives, digital versatile discs (DVD), or other optical disc storage; magnetic cassettes, magnetic tape, magnetic disc storage, or other magnetic storage devices; solid state memory technologies such as EEPROM or flash memory; or other memory technology or any other medium which can be used to store data without requiring power to retain the data after it is written. Non-volatile data storage devices 50 may be non-removable from computing device 10 as in the case of internal hard drives, removable from computing device 10 as in the case of external USB hard drives, or a combination thereof, but computing device will typically comprise one or more internal, non- removable hard drives using either magnetic disc or solid state memory technology. Non-volatile data storage devices 50 may be implemented using various technologies, including hard disk drives (HDDs) and solid-state drives (SSDs). HDDs use spinning magnetic platters and read/write heads to store and retrieve data, while SSDs use NAND flash memory. SSDs offer faster read/write speeds, lower latency, and better durability due to the lack of moving parts, while HDDs typically provide higher storage capacities and lower cost per gigabyte. NAND flash memory comes in different types, such as Single-Level Cell (SLC), Multi-Level Cell (MLC), Triple-Level Cell (TLC), and Quad-Level Cell (QLC), each with trade-offs between performance, endurance, and cost. Storage devices connect to the computing device 10 through various interfaces, such as SATA, NVMe, and PCIe. SATA is the traditional interface for HDDs and SATA SSDs, while NVMe (Non-Volatile Memory Express) is a newer, high-performance protocol designed for SSDs connected via PCIe. PCle SSDs offer the highest performance due to the direct connection to the PCIe bus, bypassing the limitations of the SATA interface. Other storage form factors include M.2 SSDs, which are compact storage devices that connect directly to the motherboard using the M.2 slot, supporting both SATA and NVMe interfaces. Additionally, technologies like Intel Optane memory combine 3D XPoint technology with NAND flash to provide high-performance storage and caching solutions. Non-volatile data storage devices 50 may be non-removable from computing device 10, as in the case of internal hard drives, removable from computing device 10, as in the case of external USB hard drives, or a combination thereof. However, computing devices will typically comprise one or more internal, non-removable hard drives using either magnetic disc or solid-state memory technology. Non-volatile data storage devices 50 may store any type of data including, but not limited to, an operating system 51 for providing low-level and mid-level functionality of computing device 10, applications 52 for providing high-level functionality of computing device 10, program modules 53 such as containerized programs or applications, or other modular content or modular programming, application data 54, and databases 55 such as relational databases, non-relational databases, object oriented databases, NoSQL databases, vector databases, knowledge graph databases, key-value databases, document oriented data stores, and graph databases.

Applications (also known as computer software or software applications) are sets of programming instructions designed to perform specific tasks or provide specific functionality on a computer or other computing devices. Applications are typically written in high-level programming languages such as C, C++, Scala, Erlang, GoLang, Java, Scala, Rust, and Python, which are then either interpreted at runtime or compiled into low-level, binary, processor- executable instructions operable on processors 20. Applications may be containerized so that they can be run on any computer hardware running any known operating system. Containerization of computer software is a method of packaging and deploying applications along with their operating system dependencies into self-contained, isolated units known as containers. Containers provide a lightweight and consistent runtime environment that allows applications to run reliably across different computing environments, such as development, testing, and production systems facilitated by specifications such as containerd.

The memories and non-volatile data storage devices described herein do not include communication media. Communication media are means of transmission of information such as modulated electromagnetic waves or modulated data signals configured to transmit, not store, information. By way of example, and not limitation, communication media includes wired communications such as sound signals transmitted to a speaker via a speaker wire, and wireless communications such as acoustic waves, radio frequency (RF) transmissions, infrared emissions, and other wireless media.

External communication devices 70 are devices that facilitate communications between computing device and either remote computing devices 80, or cloud-based services 90, or both. External communication devices 70 include, but are not limited to, data modems 71 which facilitate data transmission between computing device and the Internet 75 via a common carrier such as a telephone company or internet service provider (ISP), routers 72 which facilitate data transmission between computing device and other devices, and switches 73 which provide direct data communications between devices on a network or optical transmitters (e.g., lasers). Here, modem 71 is shown connecting computing device 10 to both remote computing devices 80 and cloud-based services 90 via the Internet 75. While modem 71, router 72, and switch 73 are shown here as being connected to network interface 42, many different network configurations using external communication devices 70 are possible. Using external communication devices 70, networks may be configured as local area networks (LANs) for a single location, building, or campus, wide area networks (WANs) comprising data networks that extend over a larger geographical area, and virtual private networks (VPNs) which can be of any size but connect computers via encrypted communications over public networks such as the Internet 75. As just one exemplary network configuration, network interface 42 may be connected to switch 73 which is connected to router 72 which is connected to modem 71 which provides access for computing device 10 to the Internet 75. Further, any combination of wired 77 or wireless 76 communications between and among computing device 10, external communication devices 70, remote computing devices 80, and cloud-based services 90 may be used. Remote computing devices 80, for example, may communicate with computing device through a variety of communication channels 74 such as through switch 73 via a wired 77 connection, through router 72 via a wireless connection 76, or through modem 71 via the Internet 75. Furthermore, while not shown here, other hardware that is specifically designed for servers or networking functions may be employed. For example, secure socket layer (SSL) acceleration cards can be used to offload SSL encryption computations, and transmission control protocol/internet protocol (TCP/IP) offload hardware and/or packet classifiers on network interfaces 42 may be installed and used at server devices or intermediate networking equipment (e.g., for deep packet inspection).

In a networked environment, certain components of computing device 10 may be fully or partially implemented on remote computing devices 80 or cloud-based services 90. Data stored in non-volatile data storage device 50 may be received from, shared with, duplicated on, or offloaded to a non-volatile data storage device on one or more remote computing devices 80 or in a cloud computing service 92. Processing by processors 20 may be received from, shared with, duplicated on, or offloaded to processors of one or more remote computing devices 80 or in a distributed computing service 93. By way of example, data may reside on a cloud computing service 92, but may be usable or otherwise accessible for use by computing device 10. Also, certain processing subtasks may be sent to a microservice 91 for processing with the result being transmitted to computing device 10 for incorporation into a larger processing task. Also, while components and processes of the exemplary computing environment are illustrated herein as discrete units (e.g., OS 51 being stored on non-volatile data storage device 51 and loaded into system memory 35 for use) such processes and components may reside or be processed at various times in different components of computing device 10, remote computing devices 80, and/or cloud-based services 90. Also, certain processing subtasks may be sent to a microservice 91 for processing with the result being transmitted to computing device 10 for incorporation into a larger processing task. Infrastructure as Code (IaaC) tools like Terraform can be used to manage and provision computing resources across multiple cloud providers or hyperscalers. This allows for workload balancing based on factors such as cost, performance, and availability. For example, Terraform can be used to automatically provision and scale resources on AWS spot instances during periods of high demand, such as for surge rendering tasks, to take advantage of lower costs while maintaining the required performance levels. In the context of rendering, tools like Blender can be used for object rendering of specific elements, such as a car, bike, or house. These elements can be approximated and roughed in using techniques like bounding box approximation or low-poly modeling to reduce the computational resources required for initial rendering passes. The rendered elements can then be integrated into the larger scene or environment as needed, with the option to replace the approximated elements with higher-fidelity models as the rendering process progresses.

In an implementation, the disclosed systems and methods may utilize, at least in part, containerization techniques to execute one or more processes and/or steps disclosed herein. Containerization is a lightweight and efficient virtualization technique that allows you to package and run applications and their dependencies in isolated environments called containers. One of the most popular containerization platforms is containerd, which is widely used in software development and deployment. Containerization, particularly with open-source technologies like containerd and container orchestration systems like Kubernetes, is a common approach for deploying and managing applications. Containers are created from images, which are lightweight, standalone, and executable packages that include application code, libraries, dependencies, and runtime. Images are often built from a containerfile or similar, which contains instructions for assembling the image. Containerfiles are configuration files that specify how to build a container image. Systems like Kubernetes natively support containerd as a container runtime. They include commands for installing dependencies, copying files, setting environment variables, and defining runtime configurations. Container images can be stored in repositories, which can be public or private. Organizations often set up private registries for security and version control using tools such as Harbor, JFrog Artifactory and Bintray, GitLab Container Registry, or other container registries. Containers can communicate with each other and the external world through networking. Containerd provides a default network namespace, but can be used with custom network plugins. Containers within the same network can communicate using container names or IP addresses.

Remote computing devices 80 are any computing devices not part of computing device 10. Remote computing devices 80 include, but are not limited to, personal computers, server computers, thin clients, thick clients, personal digital assistants (PDAs), mobile telephones, watches, tablet computers, laptop computers, multiprocessor systems, microprocessor based systems, set-top boxes, programmable consumer electronics, video game machines, game consoles, portable or handheld gaming units, network terminals, desktop personal computers (PCs), minicomputers, mainframe computers, network nodes, virtual reality or augmented reality devices and wearables, and distributed or multi-processing computing environments. While remote computing devices 80 are shown for clarity as being separate from cloud-based services 90, cloud-based services 90 are implemented on collections of networked remote computing devices 80.

Cloud-based services 90 are Internet-accessible services implemented on collections of networked remote computing devices 80. Cloud-based services are typically accessed via application programming interfaces (APIs) which are software interfaces which provide access to computing services within the cloud-based service via API calls, which are pre-defined protocols for requesting a computing service and receiving the results of that computing service. While cloud-based services may comprise any type of computer processing or storage, three common categories of cloud-based services 90 are serverless logic apps, microservices 91, cloud computing services 92, and distributed computing services 93.

Microservices 91 are collections of small, loosely coupled, and independently deployable computing services. Each microservice represents a specific computing functionality and runs as a separate process or container. Microservices promote the decomposition of complex applications into smaller, manageable services that can be developed, deployed, and scaled independently. These services communicate with each other through well-defined application programming interfaces (APIs), typically using lightweight protocols like HTTP, protobuffers, gRPC or message queues such as Kafka. Microservices 91 can be combined to perform more complex or distributed processing tasks. In an embodiment, Kubernetes clusters with containerized resources are used for operational packaging of system.

Cloud computing services 92 are delivery of computing resources and services over the Internet 75 from a remote location. Cloud computing services 92 provide additional computer hardware and storage on as-needed or subscription basis. Cloud computing services 92 can provide large amounts of scalable data storage, access to sophisticated software and powerful server-based processing, or entire computing infrastructures and platforms. For example, cloud computing services can provide virtualized computing resources such as virtual machines, storage, and networks, platforms for developing, running, and managing applications without the complexity of infrastructure management, and complete software applications over public or private networks or the Internet on a subscription or alternative licensing basis, or consumption or ad-hoc marketplace basis, or combination thereof.

Distributed computing services 93 provide large-scale processing using multiple interconnected computers or nodes to solve computational problems or perform tasks collectively. In distributed computing, the processing and storage capabilities of multiple machines are leveraged to work together as a unified system. Distributed computing services are designed to address problems that cannot be efficiently solved by a single computer or that require large-scale computational power or support for highly dynamic compute, transport or storage resource variance or uncertainty over time requiring scaling up and down of constituent system resources. These services enable parallel processing, fault tolerance, and scalability by distributing tasks across multiple nodes.

Although described above as a physical device, computing device 10 can be a virtual computing device, in which case the functionality of the physical components herein described, such as processors 20, system memory 30, network interfaces 40, NVLink or other GPU-to-GPU high bandwidth communications links and other like components can be provided by computer- executable instructions. Such computer-executable instructions can execute on a single physical computing device, or can be distributed across multiple physical computing devices, including being distributed across multiple physical computing devices in a dynamic manner such that the specific, physical computing devices hosting such computer-executable instructions can dynamically change over time depending upon need and availability. In the situation where computing device 10 is a virtualized device, the underlying physical computing devices hosting such a virtualized computing device can, themselves, comprise physical components analogous to those described above, and operating in a like manner. Furthermore, virtual computing devices can be utilized in multiple layers with one virtual computing device executing within the construct of another virtual computing device. Thus, computing device 10 may be either a physical computing device or a virtualized computing device within which computer-executable instructions can be executed in a manner consistent with their execution by a physical computing device. Similarly, terms referring to physical components of the computing device, as utilized herein, mean either those physical components or virtualizations thereof performing the same or equivalent functions.

The skilled person will be aware of a range of possible modifications of the various aspects described above. Accordingly, the present invention is defined by the claims and their equivalents.

What is claimed is:

1. A system for producing distilled spirits using an integrated intelligent control system, comprising:
a computing device comprising at least a memory and a processor;
a plurality of programming instructions that, when operating on the processor, cause the computing device to:
control a fermentation unit with controlled environmental conditions;
operate a distillation apparatus combining traditional and vacuum distillation;
use an accelerated aging unit to accelerate the aging of a distillate product;
continuously analyze chemical composition data using real-time analytical instruments;
optimize production processes using artificial intelligence algorithms;
provide a flexible production interface for customizing spirit profiles;
monitor and adjust production parameters in real-time based on data analysis;
generate comprehensive chemical fingerprints for produced spirits;
compare the generated fingerprints of produced spirit profiles against known profiles for quality assurance and authenticity verification; and
recommend modifications to production parameters to achieve desired flavor profiles.

2. The system of claim 1, wherein the adaptive spirit aging system comprises at least one of an ultrasonic aging device, a rapid thermal cycling chamber, and a packed bed bioreactor.

3. The system of claim 1, wherein the real-time analysis and response system includes analytical instrumentation capable of performing comprehensive chemical analysis for real-time monitoring of spirit composition throughout the production process.

4. The system of claim 1, wherein a dynamic flavor prediction and control system uses machine learning algorithms to predict flavor outcomes based on production parameters.

5. The system of claim 1, wherein the dynamic flavor prediction and control system further comprises a message queue subsystem.

6. The system of claim 1, further comprising an alcohol removal system for creating non- alcoholic alternatives with complex flavor profiles.

7. The system of claim 1, wherein the comprehensive chemical fingerprinting system comprises:
analytical instrumentation for generating a comprehensive chemical profile of a spirit;
a pattern recognition subsystem for identifying specific patterns in the chemical profile;
an ensemble subsystem for combining multiple analytical models;
a Bayesian subsystem for applying probabilistic modeling to the data; and
a bridging system for integrating data from multiple analytical techniques.

8. The system of claim 1, wherein the real-time analysis and response system comprises a data management subsystem and an adaptive predictive modeling subsystem.

9. A method for producing distilled spirits using an integrated intelligent control system, comprising:
a computing device comprising at least a memory and a processor;
a plurality of programming instructions that, when operating on the processor, cause the computing device to:
control fermentation of a mash under conditions optimized by adaptive control systems;
operate distillation of the fermented mash using a combination of traditional and vacuum distillation;
manage accelerated aging of the spirit using at least one novel aging technique;
continuously monitor the chemical composition of the spirit during production;
analyze monitoring data and adjust production parameters in real-time;
generate a comprehensive chemical profile of the final product; and
compare the final product's comprehensive chemical profile to a database of known profiles for quality assurance.

10. The method of claim 9, further comprising using a continuous flow system in at least one of the fermentation, distillation, or aging processes.

11. The method of claim 9, wherein the adaptive spirit aging system includes exposing spirit to ultrasonic waves or rapid thermal cycling.

12. The method of claim 9, further comprising creating a non-alcoholic version of spirit using an alcohol removal system by:
removing alcohol from the spirit;
extracting key flavor compounds;
reconstructing the flavor profile using machine learning models; and
enhancing the texture to mimic the mouthfeel of alcoholic spirits.

13. A system for analyzing and authenticating distilled spirits, comprising:
a computing device comprising at least a memory and a processor;
a plurality of programming instructions that, when operating on the processor, cause the computing device to:
maintain a database of comprehensive chemical compound profiles for mature spirits;
control analytical instruments to generate a comprehensive chemical profile of a test spirit;
execute a comparison algorithm to match the test spirit's profile against the database;
employ an artificial intelligence subsystem to interpret the comparison results; and
determine the authenticity or maturity of the test spirit based on the interpretation.

14. A method for creating custom spirit flavor profiles, comprising:
a computing device comprising at least a memory and a processor;
a plurality of programming instructions that, when operating on the processor, cause the computing device to:
receive input of desired flavor characteristics into a flavor prediction model;
generate suggested production parameters based on the input;
control production of a small batch of spirit using the suggested parameters;
analyze the chemical composition and flavor profile of the produced spirit;
feed the analysis results back into the flavor prediction model for refinement;
iterate the process until the desired flavor profile is achieved; and
output the final production parameters for the achieved flavor profile.
